(19) United States
(12) Patent Application Publication
(10) Pub. No.: US 2023/0320861 A1
(43) Pub. Date: Oct. 12, 2023

(54) SURGICAL METHODS FOR PROCEDURES OF THE SUBTALAR JOINT

(71) Applicant: Paragon 28, Inc., Englewood, CO (US)

(72) Inventors: Andrew Julian GOLDBERG, Radlett (GB); Kevin Alan DODD, Round Rock, TX (US); Richard Matthew OBERT, Poway, CA (US); Michael Edward CARROLL, Memphis, TN (US); Mathew Richard ANDERLE, Greenwood Village, CO (US); Alyssa Marie SENZ, Golden, CO (US); Somair Ali TAJ, Lakewood, CO (US); Mark MYERSON, Englewood, CO (US); Charles SALTZMAN, Salt Lake City, UT (US); Albert BURSTEIN, University Park, FL (US)

(73) Assignee: Paragon 28, Inc., Englewood, CO (US)

(21) Appl. No.: 18/298,027

(22) Filed: Apr. 10, 2023

Related U.S. Application Data
(60) Provisional application No. 63/362,850, filed on Apr. 12, 2022.

(51) Int. Cl. A61F 2/42; A61B 17/17

(52) U.S. Cl. CPC A61F 2/4202; A61B 17/1775; A61F 2002/4207

(57) ABSTRACT

A method for performing an arthroplasty of a subtalar joint includes making at least one incision adjacent the subtalar joint, accessing the subtalar joint defined superiorly by a first articular surface and inferiorly by a second articular surface, and preparing at least one of the first articular surface and the second articular surface to form first and second interfacing surfaces. The method further includes identifying at least one landmark of the first interfacing surface and the second interfacing surface, determining at least one significant point on at least one of the first interfacing surface and the second interfacing surface relative to the at least one landmark, and placing at least one trial implant. The method also includes securing an implant positioned substantially inferior relative to the first interfacing surface and substantially superior relative to the second interfacing surface, stabilizing the subtalar joint, and closing the at least one incision.

### CROSS REFERENCE TO RELATED APPLICATION

This application is a U.S. Non-Provisional Patent Application which claims priority benefit under 35 U.S.C. § 119(e) of U.S. Provisional Patent Application No. 63/362,850, filed Apr. 12, 2022, and entitled “Surgical Methods for Procedures of the Subtalar Joint,” the disclosure of which is hereby incorporated herein by reference in its entirety.

### TECHNICAL FIELD

The present disclosure relates to methods, devices, and instruments associated with performing arthroplasty procedures. The present disclosure relates to podiatric and orthopedic implants and surgery related to arthroplasty, arthrodesis, and/or arthroeresis of joints in the foot/ankle and/or procedures incorporating surrounding bones/soft tissue. More specifically, but not exclusively, the present disclosure relates to methods, implants, devices, and instruments relating to performing arthroplasty of the subtalar joint.

### BACKGROUND OF THE INVENTION

Many currently available methods, implants, instrumentation, devices, and systems for addressing joint trauma (acute and chronic, e.g., defect, gradual deterioration, etc.) do not completely address the needs of patients. Additionally, many currently available methods, implants, instrumentation, devices, and systems, for addressing joint trauma fail to account for properties of joint anatomy and associated mechanical and kinematic movement patterns/capabilities.

### SUMMARY OF THE INVENTION

The present disclosure is directed toward implants, devices, and methods for use in maintaining, correcting and/or resurfacing joint surfaces.

A first aspect of the present disclosure is a method for performing arthroplasty of a subtalar joint. The method includes making at least one incision adjacent the subtalar joint, and accessing at least a portion of the subtalar joint, wherein the subtalar joint comprises a joint space defined superiorly by a first articular surface and inferiorly by a second articular surface. The method also includes preparing at least one of the first articular surface and the second articular surface to form a first interfacing surface and a second interfacing surface, identifying at least one landmark of the first interfacing surface and the second interfacing surface; and determining at least one significant point on at least one of the first interfacing surface and the second interfacing surface relative to the at least one landmark. The method also includes analyzing the joint space by placing at least one trial implant at least partially within the joint space, securing an implant such that the implant is positioned substantially inferior relative to the first interfacing surface and substantially superior relative to the second interfacing surface, stabilizing the subtalar joint, and closing the at least one incision.

According to the first aspect of the present disclosure, the method also includes positioning the trial implant such that at least a portion of the trial implant contacts the first and second interfacing surfaces.

According to the first aspect of the present disclosure, the method also includes placing a second implant trial at least partially within the joint space, wherein the implant trial comprised a first size and the second implant trial comprises a second size, wherein the second size is different from the first size.

According to the first aspect of the present disclosure, the method also includes performing a fibular osteotomy to access at least a portion of at least one of the subtalar joint and the joint space.

According to the first aspect of the present disclosure, the method also includes analyzing a first position and a first function of the implant after the implant has been placed within at least a portion of the joint space.

According to the first aspect of the present disclosure, the method also includes temporarily fixating at least one of a calcaneum and a talus prior to preparing at least one of the first and second articular surfaces.

According to the first aspect of the present disclosure, the method also includes adjusting the implant within at least a portion of the joint space such that the implant assumes a second position and a second function, wherein the second position and function are different than the first position and function.

According to the first aspect of the present disclosure, the method also includes stabilizing the subtalar joint by manipulating one or more soft tissue structures adjacent the subtalar implant after the implant has been placed and fixated at least partially within the joint space to reach a first stability.

According to the first aspect of the present disclosure, the method also includes confirming stability of the subtalar joint.

According to the first aspect of the present disclosure, the method also includes securing the implant by placing at least a first implant component adjacent the first interfacing surface and placing a second implant component adjacent the second interfacing surface.

According to the first aspect of the present disclosure, the method also includes placing a third implant component at least partially within the joint space such that the third implant component is positioned substantially inferior relative to the first implant component and substantially superior relative to the second implant component.

According to the first aspect of the present disclosure, the method also includes the at least one significant point being least one center of rotation disposed on the second interfacing surface.

According to the first aspect of the present disclosure, the method also includes aligning one or more components of the subtalar joint.

According to the first aspect of the present disclosure, the method also includes aligning the subtalar joint by manipulating at least one of a talus and a calcaneum such that at least a head of the talus is disposed substantially superior relative to at least a portion of the calcaneum.

According to the first aspect of the present disclosure, the method also includes the at least one landmark being one or more facets substantially disposed on a superior surface of the calcaneum.

According to the first aspect of the present disclosure, the method also includes the at least one landmark being one or more facets substantially disposed on a superior surface of the calcaneum.

According to the first aspect of the present disclosure, the method also includes the first interfacing surface being an inferior surface of the talus and the second interfacing surface being a superior surface of the calcaneum.

According to the first aspect of the present disclosure, the method also includes manipulating one or more soft tissue structures.

A second aspect of the present disclosure is a method for performing an arthroplasty of a subtalar joint. The method includes accessing at least a portion of the subtalar joint, identifying at least one landmark of at least one of an inferior surface and a superior surface, and determining at least one center of rotation disposed on at least one of the inferior and superior surfaces. The method also includes placing at least one trial implant adjacent the inferior surface and the superior surface, and securing an implant such that the implant is positioned substantially between the inferior and superior surfaces.

A third aspect of the present disclosure is a method for performing an arthroplasty of a subtalar joint including a first surface and a second surface. The method includes identifying at least one landmark of at least one of the first surface and the second surface, determining at least one significant point in relation to the at least one landmark, and securing an implant between the first and second surfaces in a first position, wherein the first position is based on a position of the at least one significant point.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a top view of a calcaneus shown in the transverse plane, in accordance with the present disclosure;

FIG. 2 is a bottom view of a talus shown in the transverse plane, in accordance with the present disclosure;

FIG. 3 is a top view of skeletal structures of the midfoot and hindfoot, in accordance with the present disclosure;

FIG. 4 is a top view of skeletal structures of the midfoot and hindfoot, in accordance with the present disclosure;

FIG. 5 is a top view of skeletal structures of the midfoot and hindfoot, in accordance with the present disclosure;

FIG. 6 is a top view of skeletal structures of the midfoot and hindfoot, in accordance with the present disclosure;

FIG. 7 is a flowchart of an aspect of an exemplary method for performing an arthroplasty of a subtalar joint, in accordance with the present disclosure;

FIG. 8 is a flowchart of a first exemplary sub-method associated with the exemplary method of performing a subtalar arthroplasty shown in FIG. 7, in accordance with the present disclosure;

FIG. 9 is a flowchart of a second exemplary sub-method associated with the exemplary method of performing a subtalar arthroplasty shown in FIG. 7, in accordance with the present disclosure;

FIG. 10 is a flowchart of a third exemplary sub-method associated with the exemplary method of performing a subtalar arthroplasty shown in FIG. 7, in accordance with the present disclosure;

FIG. 11 is a flowchart of a fourth exemplary sub-method associated with the exemplary method of performing a subtalar arthroplasty shown in FIG. 7, in accordance with the present disclosure;

FIG. 12 is a flowchart of an aspect of an exemplary method for performing arthroplasty of a subtalar joint, in accordance with the present disclosure;

FIG. 13 is a flowchart of a first exemplary sub-method associated with the exemplary method of performing a subtalar arthroplasty shown in FIG. 12, in accordance with the present disclosure;

FIG. 14 is a flowchart of a second exemplary sub-method associated with the exemplary method of performing a subtalar arthroplasty shown in FIG. 12, in accordance with the present disclosure;

FIG. 15 is a flowchart of a third exemplary sub-method associated with the exemplary method of performing a subtalar arthroplasty shown in FIG. 12, in accordance with the present disclosure; and

FIG. 16 is a flowchart of a fourth exemplary sub-method associated with the exemplary method of performing a subtalar arthroplasty shown in FIG. 12, in accordance with the present disclosure.

### DETAILED DESCRIPTION

In this detailed description and the following claims, the words proximal, distal, anterior, or plantar, posterior, or dorsal, medial, lateral, superior, and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation, and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

The instruments, implants, systems, assemblies, and related methods for maintaining, correcting, and/or resurfacing joint surfaces of the present disclosure may be similar to, such as include at least one feature or aspect of, the implants, methods, devices, and/or components as disclosed in U.S. Pat. No. 10,117,749, issued on Nov. 6, 2018 and entitled “Subtalar Joint Implant”; European Patent No. 3756626 issued on Dec. 30, 2020 and entitled “Subtalar Joint Implant”;, European Patent Application No. 15770960.1A filed on Jul. 15, 2020 and entitled “Subtalar Joint Implant”; U.S. patent application Ser. No. 17/653,029, filed on Mar. 1, 2022 and entitled “Methods for Performing an Arthroplasty of the Subtalar Joint”; and U.S. Provisional Patent Application No. 63/155,100 filed on Apr. 2, 2021 and entitled “Systems and Methods for Controlled Facet Repositioning in the Calcaneus”; and U.S. Non-Provisional application Ser. No. 17/657,522 filed on Mar. 31, 2022 which are all hereby incorporated herein by reference in their entireties.

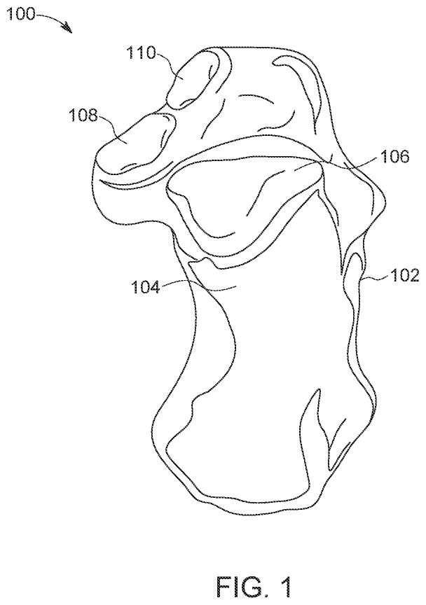

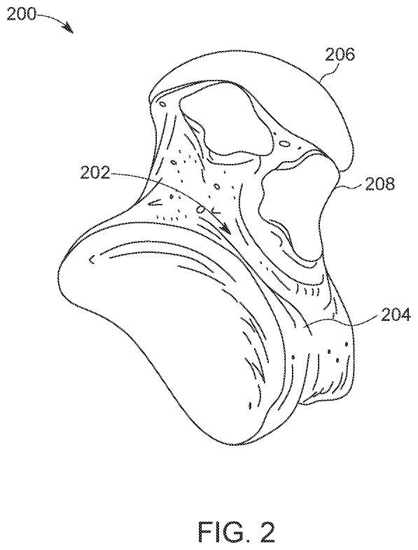

Referring to the drawings, wherein like reference numerals are used to indicate like or analogous components throughout the several views, and with particular reference to FIGS. 1-2, there is illustrated an exemplary embodiment of a right calcaneum (e.g., calcaneus) 100 shown from a superior view and a right talus 200 shown from an inferior view. The calcaneum 100 and the talus 200 are bones of the foot/ankle and two bones of the subtalar joint which is disposed substantially inferior (e.g., distal) relative to the ankle. According to some research and corresponding literature, the subtalar joint may be considered two joints based on one or more facets disposed on the calcaneum 100, which are shown with reference to FIG. 1 and described subsequently herein. These two joints include a first joint (the subtalar joint) including the posterior facet of the calcaneum 100, and a second joint (the talocalcaneonavicular joint) including the middle and/or anterior facet as well as the talonavicular joint (including the talus 200 and the navicular (not shown)). However, for the purpose of this disclosure the subtalar joint is considered to include at least the calcaneum 100 (including the posterior, middle, and anterior facets), the talus 200, and the soft tissue disposed therebetween. The subtalar joint, which is positioned superior relative to the calcaneum 100 and inferior relative to the talus 200 is configured to permit/enable movement. Typically, the subtalar joint permits movement that includes at least inversion and eversion of the foot, where inversion is defined as a movement that causes the soles of the feet to face inwards (e.g., in a medial direction) and eversion is defined as a movement that causes the soles of the feet to face outwards (e.g., laterally). Further, inversion and eversion can include external and internal rotation, respectively, of the talus 200 relative to (e.g., on) the calcaneum 100. The calcaneum 100 and the talus 200 (and components thereof) are shown and described herein so as to provide context for various steps of the subsequently disclosed method of performing arthroplasty of the subtalar joint. Furthermore, it should be noted that the calcaneum 100 and the talus 200 as shown, correspond to such bones found in the right foot of a human and that the structures shown and described herein may have different sizing and/or orientation with respect to the left foot of a human (but can be reasonably assumed to be equal and opposite) in the medial-lateral directions. It should be understood that the calcaneum 100 and the talus 200 may not be representative of the geometry of such bones of any/all humans/patients but are shown herein to be representative of the general geometry and features of the normally configured bones. For example, trauma, bone deformity, arthritis and other conditions can alter the geometry of such bones. As referred to herein, arthroplasty is defined as surgical reconstruction and/or joint replacement of a joint (in the context of this disclosure, the subtalar joint). It should be known that both the calcaneum 100 and the talus 200 may include other geometric features, interfaces with other portions of anatomy, and articulations in addition to those discussed herein.

The calcaneum 100 is shown to include a body 102 which includes a top surface 104. The top surface 104 of the calcaneum 100 may substantially include a superior surface of the calcaneum 100 and form at least a portion of an inferior portion and/or defining surface of the subtalar joint. In some aspects, the top surface 104 interfaces with cartilage positioned between the calcaneum 100 and the talus 200 (e.g., the subtalar joint) that may be removed in performing a subtalar joint arthroplasty. In some aspects, the top surface 104 may become one of two interfacing surfaces of a subtalar joint after arthroplasty is performed (e.g., a surface that interfaces with a subtalar joint implant). The top surface 104 may have various geometries depending on the patient and the condition of the calcaneum 100 (e.g., trauma, arthritis, deformity, etc.). As shown, the top surface 104 includes a posterior facet 106, a middle facet 108, and an anterior facet 110 as shown in FIG. 1. In some aspects, one or more of the posterior facet 106, the middle facet 108 and the anterior facet 110 may be less distinguishable than shown on the calcaneum 100 in FIG. 1. For example, in some aspects the middle facet 108 and the anterior facet 110 may be positioned closer to one another than shown in FIG. 1 such that the middle facet 108 and the anterior facet 110 overlap and/or abut one another (e.g., there is not a gap positioned anterior-lateral the middle facet 108). In some aspects, the calcaneum body 102, the posterior facet 106, the middle facet 108, the anterior facet 110 (as well as other components of the top surface 204 and the calcaneum 100) may be referenced as anthropometric markers (e.g., landscape markers, identifiers, etc.) in order to analyze movement and/or other kinematic properties of the calcaneum 100 and/or surrounding joints. It should also be understood that one or more events or conditions (e.g., trauma, arthritis, deformity, etc.) may alter the calcaneum 100 of a patient such that one or more of the features described has an altered geometry, is damaged, or is not present on the calcaneum 100 of a patient.

The talus 200 is shown to include a body 202 and a bottom surface 204. The top surface 204 of the talus 200 may substantially include an inferior surface of the talus 200 and form at least a portion of a superior portion and/or defining surface of the subtalar joint. In some aspects, the bottom surface 204 interfaces with cartilage positioned between the calcaneum 100 and the talus 200 (e.g., the subtalar joint) that may be removed in performing a subtalar joint arthroplasty. In performing an arthroplasty of the subtalar joint, at least a portion of subcortical bone may be removed in order to place one or more components of an implant so as to promote maximum stability of the joint as well as other surrounding joints and structures. In some aspects, the bottom surface 204 may become one of two interfacing surfaces of a subtalar joint after arthroplasty is performed (e.g., a surface that interfaces with a subtalar joint implant). The bottom surface 204 may have various geometries depending on the patient and the condition of the calcaneum 100 (e.g., trauma, arthritis, deformity, etc.). The talus 200 is further shown to include a talar head 206 positioned at an anterior portion of the talus 200, with a talar neck 208 extending between the body 202 and the talar head 206. In some aspects, the talar body 202, the talar head 206, and the talar neck 208 (as well as other components of the bottom surface 204 and the talus 200) may be referenced as anthropometric markers (e.g., landscape markers, identifiers, etc.) .) in order to analyze movement and/or other kinematic properties of the talus 200 and/or surrounding joints.

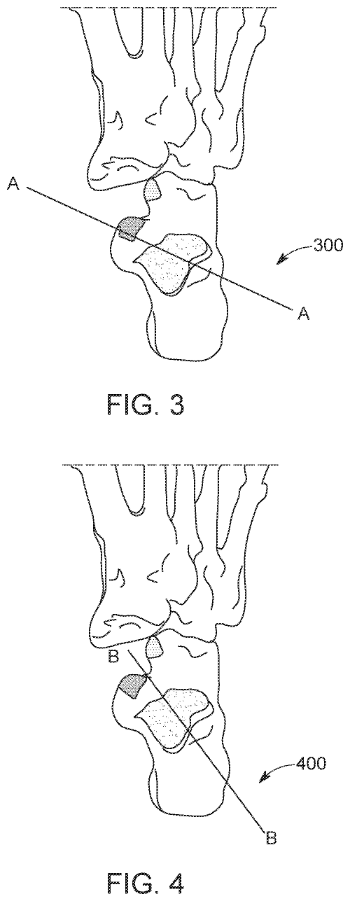

Referring to FIG. 3, an axis 300 is shown to extend along the line A-A from a point at or near (or through) a proximal portion of the middle facet to a portion at or near (or through) the posterior facet. The axis 300 may correspond to an axis of the calcaneum (e.g., an axis about which the calcaneum moves with respect to the talus). The axis 300 may correspond to a best fit cylinder created through the posterior facet and the articular geometry thereof. The axis 300 may extend from a medial prominent apex of the middle facet passing through a prominent point on the medial portion of the posterior facet and terminating near a prominent point on the middle facet.

Referring to FIG. 4, an axis 400 is shown to extend along the line B-B from a point at or near (or through) a distal portion of the middle facet to a portion at or near (or through) the posterior facet. The axis 400 may correspond to an axis of the calcaneum (e.g., an axis about which the calcaneum moves with respect to the talus). The axis 400 may correspond to a best fit cylinder through the articular geometry of the posterior facet. The axis 400 may extend between a medial and posterior prominent apex of the posterior facet to and/or through and/or near a central articulating point within an acetabulum pedis defined by a talar navicular joint, the middle and anterior facets, and a spring ligament complex.

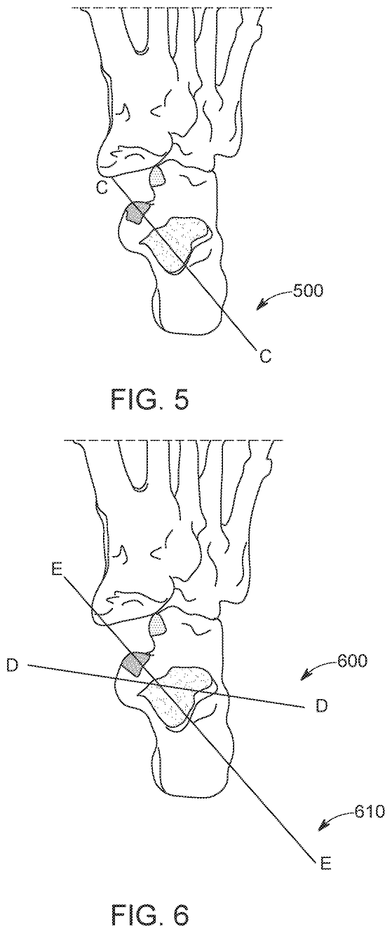

Referring to FIG. 5, an axis 500 is shown to extend along the line C-C from a point at or near (or through) a distal portion of the middle facet to a portion at or near (or through) the calcaneum. The axis 500 may also correspond to an axis of the calcaneum (e.g., an axis about which the calcaneum moves with respect to the talus). The axis 500 may correspond to one or more features/components of the articular geometry of the posterior facet. The axis 500 may extend between a posterior prominent apex of the posterior facet and extend to/pass through a prominent point on the medial portion of the posterior facet and terminate near the center of the middle facet.

Referring to FIG. 6, an axis 600 is shown to extend along the line D-D and an axis 610 is shown to extend along the line E-E. The axes 600, 610 may correspond to an axis of the calcaneum (e.g., an axis about which the calcaneum moves with respect to the talus). The axes 600, 610 may correspond to the articular geometry of the posterior facet. The axes 600, 610 may rotate during activity about an area near the interosseous ligament or the medial prominent aspect of the posterior facet. The axes 600, 610 may also correspond to a heel strike axis and a stance to toe-off axis, about which movement of the calcaneum, talus, and/or subtalar joint may occur.

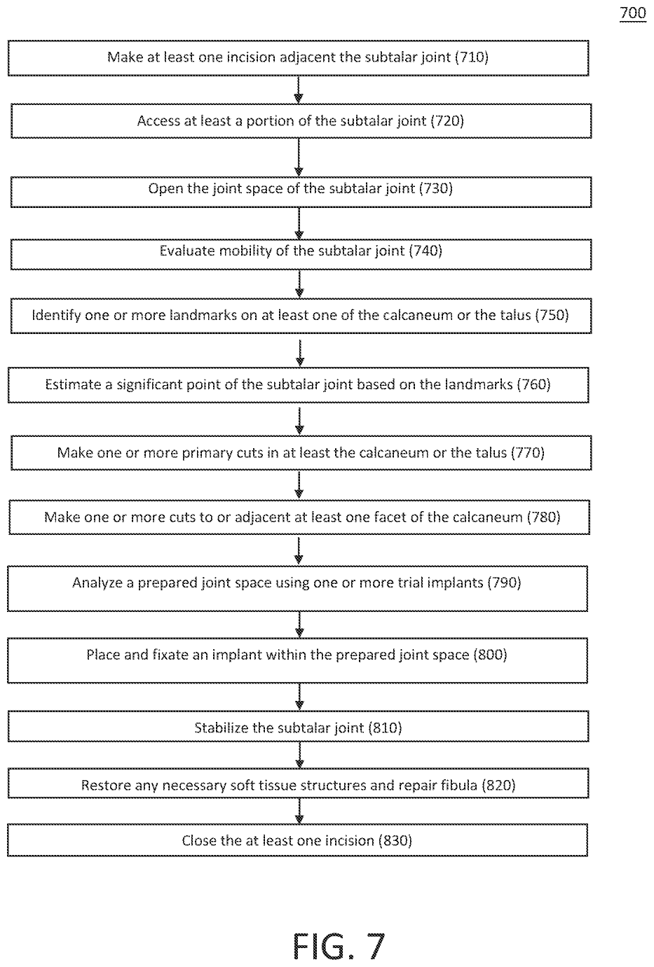

Referring now to FIGS. 7-16, there are illustrated flowcharts showing exemplary processes (e.g., methods) of performing an arthroplasty of the subtalar joint, as well as exemplary sub-processes of said exemplary processes. The steps of the processes and sub-processes shown in FIGS. 7-16 may reference one or more anatomical components, for example those shown and described with reference to FIGS. 1-6. However, it should be noted that the steps of the processes and sub-processes shown and described with reference to FIGS. 7-16 are in no way limited to the geometric features of the anatomy shown in FIGS. 1-6 and may be applied to subtalar joints and components thereof having one or more alternate geometric features to those shown and described in FIGS. 1-6.

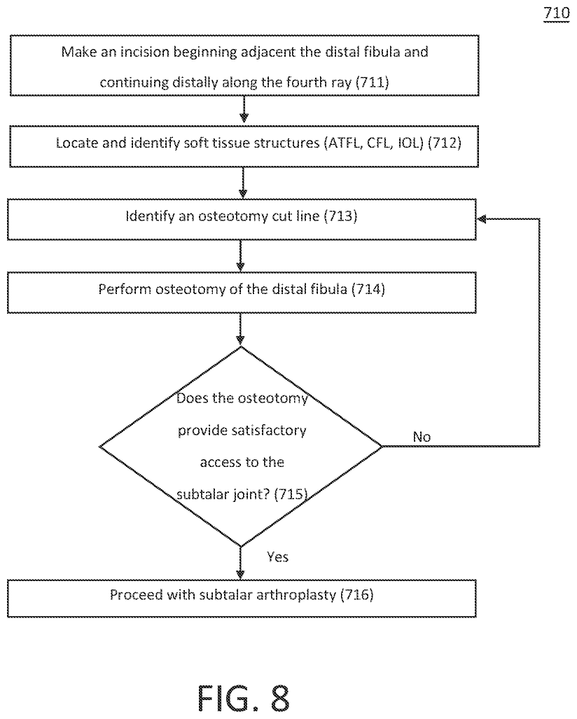

FIG. 7 shows a flowchart for an exemplary process 700 of performing a subtalar arthroplasty, according to one aspect of the present disclosure. The process 700 is further shown to include exemplary sub-steps shown and described with reference to FIGS. 8-11. Subtalar arthroplasty, as with all medical procedures, should be performed by one or more physicians, surgeons, or otherwise adequately trained healthcare providers and is shown and described as such. It should be understood that the steps of the process 700 (as well as the steps of the associated sub-processes) may be skipped, performed in an alternate order, repeated, or otherwise modified in performing a subtalar arthroplasty. In some aspects, alternate steps may be added and/or substituted for the steps shown and described herein with reference to the process 700. Furthermore, it should also be understood that the steps of the process 700 may not be limiting; that is to say that in some aspects additional steps may be performed in order to perform a subtalar arthroplasty. The steps of the process 700 may be performed with one or more of the implants, instruments, devices, or other concepts discussed in the issued patents and pending patent applications discussed previously and incorporated by reference herein in their entireties. Further, in some aspects one or more implants and/or pieces thereof may be implanted according to the process 700, for example an implant may include multiple pieces configured to interface with or adjacent to the posterior facet 106, the middle facet 108, the anterior facet 110, or other anatomical features. In some aspects, additional and/or alternative implants, instruments, devices, and/or other concepts to those incorporated by reference herein in their entireties may be implemented in conjunction with that that is shown and described herein.

The process 700 is shown to include a step 710 making at least one incision substantially adjacent a subtalar joint, according to the exemplary method shown and described in FIGS. 7-11. In some aspects, the at least one incision may be an incision extending from adjacent the distal tip of the fibula to the base of the fourth or fifth metatarsal (and/or extending along/to the fourth or fifth ray) such that the subtalar joint may be accessed laterally. However, in some aspects one or more circumstances (e.g., trauma, deformity, previous procedures, minimize surgical morbidity, etc.) may be present that are conducive to alternate incisions and/or directional approaches. For example, in some aspects the at least one incision may include an extended lateral incision/approach (e.g., in which a vertical limb meets a horizontal limb and the soft tissues on the side of the calcaneum are lifted off to access the lateral wall of the calcaneum and the subtalar joint). Additional incisions may also be made at, near, or adjacent the subtalar joint in order to provide optimal visualization of the subtalar joint and surrounding soft tissue structures. In some aspects, a subtalar arthroplasty may include removal of metalwork, for example after trauma where previous metalwork is in the calcaneum and/or talus and requires removal during a subtalar arthroplasty procedure, and accordingly, the step 710 may include making one or more incisions configured to facilitate the removal of said metalwork. Further, in some aspects other alternate incisions and/or approaches may be taken, for example a posterior-lateral approach and/or a posterior approach (which, in some aspects, may require manipulating/taking down/severing the Achilles tendon). In some aspects, incisions may be made that are not directly adjacent to the joint. Additionally, in some aspects, a patient may have one or more conditions or circumstances that warrant a medial approach, for example trauma has occurred to one or more portions of the foot, ankle, and/or lower leg that prohibit approaches/incisions in directions other than the medial direction relative to the subtalar joint. Accordingly, in such circumstances a medial incision may be made. It should be understood that the step 710 may also include multiple incisions being made which may include a combination of two or more of the aforementioned incisions and/or alternate incisions (e.g., a posterior and a medial incision, a medial and a lateral incision, etc.).

The step 710 is shown to include an exemplary sub-process as shown in FIG. 8 which, according to some aspects, includes sub-steps which may be performed in performing the step 710. It should be understood that one or multiple sub-steps of the step 710 may be omitted, repeated, or performed in an alternate order to that shown in the subprocess of FIG. 8. FIG. 8 is shown to include a sub-step 711 of making an incision adjacent the distal fibula and continuing distally along the fourth ray, according to an exemplary method. In some aspects, the incision of sub-step 711 may overlap with the step 710 in that a single incision may be made so long as the single incision suffices to provide necessary access to the subtalar joint and adjacent structures. In some aspects, the incision of the step 711 may be extended or otherwise modified based on an evaluation of access to the subtalar joint and adjacent structures following the making of the incision.

The step 710 is further shown to include a sub-step 712 of locating and identifying soft tissue structures, according to some aspects. The sub-step 712 may include the identification of one or more soft tissue structures including but not limited to the ATFL, the CFL, and the IOL. In some aspects, the sub-step 712 may include repositioning one or more soft tissue structures adjacent the subtalar joint so as to achieve access to the joint. Further, the sub-step 712 may also include severing, resecting, or otherwise modifying one or more soft tissue structures in the event that one or more of said soft tissue structures obstructs access to the subtalar joint and adjacent structure and a determination that such structure(s) cannot be repositioned.

The step 710 is shown to include a sub-step 713 of identifying an osteotomy cut line, according to some aspects. The sub-step 713 may include the implementation of pre-operative imaging, for example, CT, standing CT, x-ray, etc., and intraoperative identification or confirmation of an estimated and/or predicted osteotomy line on the distal fibula. The identification of the osteotomy cut line may include a physician marking or otherwise indicating on a distal portion of the fibula and intended cut line. In some aspects, the sub-step 713 may include the use of one or mor cut guides configured to guide a fibular osteotomy cut. For example, the sub-step 713 may include coupling a cut guide with a fibula of a patient and confirming that the cut that would be guided by said cut guide aligns with a desired cut of the physician. Further, in some aspects, the sub-step 713 may include the coupling of a patient specific instrument (e.g., PSI) cut guide that has been 3-D printed or otherwise produced specific to the anatomy of the patient. If a PSI cut guide is implemented, the sub-step 713 may include confirmation of a fit of the cut guide with the fibula and other surrounding anatomy of the patient, as well as a confirmation that the cut to be guided by the PSI cut guide corresponds to a desired cut of the physician.

The step 710 is further shown to include a sub-step 714 of performing an osteotomy of the distal fibula (e.g., a fibular osteotomy), according to some aspects. The sub-step 714 may be performed using any common orthopedic instrument (e.g., sagittal saw, reciprocating saw, and/or other common cutting/resection instruments) in conjunction with one or more cut guides including but not limited to those described with reference to sub-step 713. The sub-step 714 may include one or more cuts to the fibula being made by the physician.

The step 710 is further shown to include a sub-step 715 of evaluating if the osteotomy provides satisfactory access to the subtalar joint, according to some aspects. After performing the fibular osteotomy of the sub-step 714, the physician may evaluate the access to the subtalar joint provided by the fibular osteotomy. In some aspects, the sub-step 715 may include the physician manipulating the subtalar joint, ankle joint, and/or other anatomical structures in order to evaluate access to the subtalar joint. In the event that the physician determines that access to the subtalar joint is insufficient, the physician may repeat the sub-step 713 of identifying an osteotomy line as well as subsequent sub-steps 714 and 715 until a determination is made that the fibular osteotomy provides sufficient access to the subtalar joint. Once such a determination is made, the physician may proceed to a sub-step 716 of proceeding with the subtalar arthroplasty procedure.

Referring again to FIG. 7, the process 700 is shown to include a step 720 of accessing at least a portion of the subtalar joint, according to the exemplary method shown and described in FIGS. 7-11. The step 720 may be performed directly after the step 710 as shown and described previously, for example at least a portion of the subtalar joint may be accessed via the at least one incision made in the step 710. Further, the step 720 may in some aspects be dependent on the one or more incisions made in the step 710 (e.g., if a lateral incision was made in the step 710, then the step 720 may include accessing the subtalar joint from a lateral direction). It should also be noted that the step 720 may include the manipulation, repositioning, and/or resection of one or more soft tissue structures disposed between the one or more incision made in the step 710. For example, if a lateral incision was made in the step 710, the step 720 may include manipulating, repositioning, bluntly dissecting, etc. one or more of the calcaneofibular ligament, the anterior talofibular ligament, and/or the extensor digitorium brevis (and/or other soft tissue structures which may have been previously identified and/or preliminarily manipulated in sub-steps of the step 710 as shown and described previously herein). The soft tissue structures that may be contacted or otherwise addressed in the step 710 (including sub-steps thereof) may be dependent on the one or more incisions made in the step 710.

The process 700 is shown to include a step 730 opening a joint space of the subtalar joint, according to the exemplary method shown and described in FIGS. 7-11. The step 730 may include implementing one or more instruments common to arthroplasty procedures including, for example laminar spreaders, bone distractors over wires, elevators, osteotomes, sensors for ligament balancing, tracking arrays for bony alignment, PSI components, etc. Similar to the step 720, the step, 730 may be dependent on the one or more incisions made in the step 710 (and/or the fibular osteotomy performed in the sub-steps of the step 710). For example, the instruments and approach implemented in opening the joint space may be dependent on whether the joint is being accessed from a lateral, posterior lateral, posterior, medial, or other directional approach. Further, the step 730 and/or the instruments implemented in conjunction 730 may be dependent on the geometric features of the subtalar joint and components thereof (e.g., the calcaneum and the talus). For example, in accessing the joint laterally the physician may implement an instrument that is conducive to contact with the talus at or adjacent the sinus tarsi (disposed on the talus, but typically positioned adjacent the middle facet). Conversely, in accessing the subtalar joint posteriorly the physician may implement one or more instruments conducive to contacting the posterior facet as shown and described with reference to FIG. 1.

The process 700 is shown to include a step 740 evaluating mobility of the subtalar joint, according to the exemplary method shown and described in FIGS. 7-11. The step 740 may include the physician immobilizing and/or manipulating various anatomical structures including the subtalar joint as well as other structures adjacent the subtalar joint (e.g., tibia ankle joint, soft tissue structures, etc.). In some aspects, the step 740 may include the physician performing various range of motion (ROM) testing and measuring the ROM of the subtalar joint. In some aspects, the step 740 may result in a determination that one or more of the preceding steps of the process 700 need to be repeated, for example if the access to the subtalar joint is insufficient relative to the mobility of the subtalar joint that is evaluated in the step 740. In evaluating the ROM of the subtalar joint, the physician may record various measurements against which mobility of the subtalar joint may be compared later in the procedure/process 700 (e.g., after the arthroplasty has been performed).

The process 700 is shown to include a step 750 identifying one or more landmarks on at least one of the calcaneum or the talus, according to the exemplary method shown and described in FIGS. 7-11. The step 750 may include identifying one or more of the geometric features of the calcaneum 100 and/or the talus 200 shown and described with reference to FIGS. 1-6, for example, the posterior facet 106, the middle facet 108, the anterior facet 110, the talar head 206, the talar neck 208, etc. In some aspects the step 750 may be dependent on one or more of the previous steps of the process 700 in that the landmarks that may be identifiable on the calcaneum 100 and/or the talus 200 may be dependent on the directional approach taken by a physician (e.g., dependent on the one or more incisions made and whether said incisions were made laterally, posterior-laterally, posterior, medial, etc. relative to the subtalar joint). In some aspects, the step 750 (and subsequent step 760) may further include the implementation of one or more imaging methods. For example, fluoroscope, CT, MRI, X-ray, or other imaging technologies may be implemented in order to capture images of the calcaneum 100, the talus 200 (and/or portions thereof), and/or anatomical components adjacent the subtalar joint for analysis by one or more physicians. As mentioned previously, in some aspects one or more components of the calcaneum 100, the talus 200, or other anatomical features adjacent the subtalar joint may not be present or may not be identifiable (e.g., trauma, degeneration, deformity, etc.). In some aspects, the step 750 may include identifying one or more of the aforementioned landmarks with respect to one or more of the other aforementioned landmarks (or other anatomical landmarks entirely). For example, if the middle facet 108 and the anterior facet 110 abut one another, the posterior facet 108 may be identified as a landmark and furthermore may be identified (e.g., measured, mapped, etc.) with respect to landmarks of the calcaneum 100 other than the middle facet 108 and the anterior facet 110 or with respect to landmarks of the talus 200, for example the talar head 206. The step 750 may also include the identification and measurement/estimation of one or more angles associated with the calcaneum 100, the talus 200, the subtalar joint, or other anatomical features/structures, for example the angle of Gissane. Soft tissue landmarks may also be identified including the soft tissue structures mentioned previously as well as other soft tissue structures (which may depend on directional approach), for example the interosseous ligament, the deltoid ligament, peroneal tendons, calcaneofibular ligament, sural nerve, superficial peroneal nerve, neurovascular bundle (if medial approach) etc.

The process 700 is shown to include a step 760 estimating a significant point of the subtalar joint based on the one or more landmarks, according to the exemplary method shown and described in FIGS. 7-11. In some aspects, the step 760 may include estimating (or, in some aspects, determining) one or more significant points of the subtalar joint based on the one or more landmarks. The step 760 may further include the application of one or more algorithms, software programs, mathematical models, or other tools in order to analyze the landmarks identified (and in some cases imaged) in the step 750. Additionally, significant points of the subtalar joint may include and/or be referenced/identified with respect to one or more of the geometric features as shown and described previously with reference to FIGS. 1-6 (e.g., talar head, talar neck, one or more facets of the calcaneum, etc.). Further, other anatomical structures that may be positioned adjacent the subtalar joint (or in some research considered to be a part of the subtalar joint, for example the navicular) may also be incorporated in the identification of the one or more significant points. The significant points of the step 760 may include one or more centers of rotation, axis/axes, articulation points, pivot points (e.g., multiple pivot points along a line), one or more points along or central to a spline (e.g., piecewise polynomial function), ellipse, or other geometric curve or shape. Further, the one or more significant points of the step 760 may correspond to the aforementioned motion (e.g., inversion and eversion) of the subtalar joint. For example, the one or more significant points may correspond with one or more kinematic/mechanical properties and/or movements of the subtalar joint and, accordingly, correspond to one or more components of an implant to be implanted in a subtalar arthroplasty procedure. In some aspects, the one or more significant points may further correspond to one or more sizes of one or more components of an implant to be placed and fixated in the total arthroplasty procedure included in the process 700. For example, the location of one or more significant points (e.g., centers of rotation) estimated/determined/located on the top surface 104 of the calcaneum 100 may correspond to a size and/or placement/fixation location of one or more implant components. Similarly, one or more estimated/determined significant points and the location/proximity of said one or more significant points to one or more of the anatomic landmarks as shown and described previously may also correspond to sizing, placement, and/or selection of one or more components of an implant.

The process 700 is shown to include a step 770 making one or more primary cuts in at least one of the calcaneum and/or the talus, according to the exemplary method shown and described in FIGS. 7-11. As mentioned previously with reference to prior steps in the process 700, the step 770 may depend at least in part on the one or more incisions made in step 710. For example, if the subtalar joint is accessed laterally, step 770 may include preparing (e.g., at least making one or more cuts to) the lateral-most portions of one or more surfaces (e.g., articular, interfacing, etc.) of the calcaneum and/or the talus prior to preparing the medial-most portions. With reference to FIGS. 1-2, the top surface 104 and the bottom surface 204 of the calcaneum 100 and the talus 200, respectively, typically interface with cartilage and a subtalar arthroplasty procedure typically includes removing at least some (or in some aspects, all) of said cartilage and/or subchondral bone from the aforementioned surfaces. After the aforementioned cartilage is removed, the top surface 104 of the calcaneum 100 and the bottom surface 204 of the talus 200 (which may be further resected and/or otherwise cut in subsequent steps) may contact the majority of a subtalar implant that is implanted and fixated between the calcaneum 100 and the talus 200 (and thus interfacing with the top surface 104 and the bottom surface 204). In some aspects, the step 770 may further include implementing one or more instruments in order to modify the top surface 104 of the calcaneum 100 and/or the bottom surface 204 of the talus 200. For example, in some aspects a physician may score (e.g., create an uneven texture by creating one or more depressions in the top surface 104 and/or the bottom surface 204) so as to promote fixation of and/or further preparation of the top surface 104 and/or the bottom surface 204 for arthroplasty.

The process 700 is shown to include a step 780 making one or more cuts to or adjacent to at least one facet of the calcaneum, according to the exemplary method shown and described in FIGS. 7-11. The step 780, similar to one or more of the steps of the process 700 as described previously, may be dependent on the access direction (based on the one or more incisions of the step 710) of the subtalar joint. For example, the step 780 may include performing one or more cuts on the top surface 104 of the calcaneum at or adjacent the posterior facet 106, the middle facet 108, the anterior facet 110. The approach, angle, instrumentation (e.g., saw, blade, drill, etc.) appropriate for such cuts may be dependent on the access and approach angle of the patient and the specific arthroplasty procedure. Depending on the approach angle and the access to the calcaneum 100, different cuts and/or cutting patterns may need to be made/performed. In some aspects, a first cutting instrument may be implemented for a subtalar arthroplasty performed through a lateral incision, and a second instrument may be implemented for a different subtalar arthroplasty performed through alternate directions/access points. In some aspects, the one or more cuts of the step 780 may correspond to one or more components of an implant to be placed during a subtalar arthroplasty procedure. For example, a first cut or cuts may be made on the posterior facet 106 of the calcaneum 100 that correspond to a size, geometry, position, and function of a first implant component configured to be placed and/or fixated to/adjacent to the posterior facet 106 of the calcaneum 100. In some aspects, the step 780 may include the placement of one or more cut guides and/or jigs configured to guide one or more cuts, including, but not limited to, PSI cut guides.

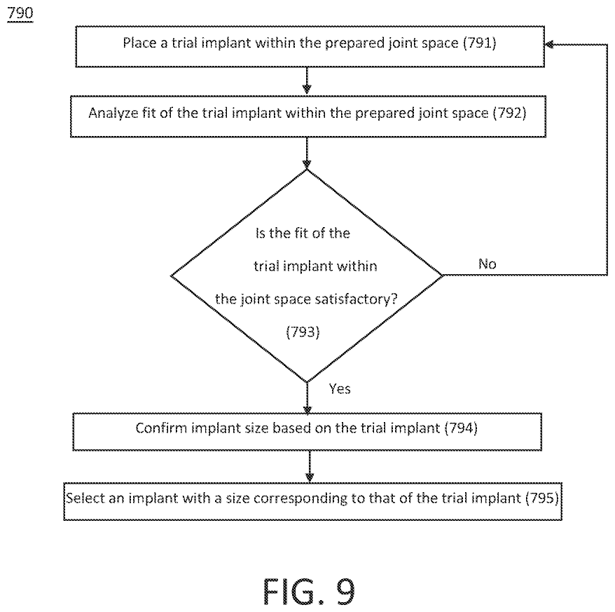

The process 700 is shown to include a step 790 analyzing a prepared joint space using one or more trial implants, according to the exemplary method shown and described in FIGS. 7-11. The step 790 may be performed one or more times and, in some aspects may be an iterative process based on feedback received by the physician or other medical professional performing the step 790. Accordingly, the step 790 is shown as a sub-process including various exemplary sub-steps shown and described with reference to FIG. 9. The step 790 may be repeated one or more times, in part or in full. As with the process 700, one or more sub-steps of the step 790 may be repeated, skipped, performed in an alternate order, or may be substituted for another step not shown in the exemplary sub-steps of the step 790 of FIG. 9.

The step 790 is shown to include a sub-step 791 placing a trial implant within the prepared joint space, according to an exemplary method/sub-process shown and described in FIG. 9. The trial implant may be placed such that at least a first portion of the trial implant is in contact with the top surface 104 of the calcaneum and at least a second portion of the trial implant is in contact with the bottom surface 204 of the talus. It should be noted that in some aspects, trial implants may be placed both before and after cuts are made, surfaces are prepared, etc. In some aspects, the sub-step 791 may include placing/securing/positioned one or more components of the trial implant relative to the one or more significant points identified in the step 750. For example, the sub-step 791 may include placing a trial implant with features that correspond to one or more centers of rotation, pivot points, or other points included on an implant that correspond to a mechanism configured to mimic motion of a healthy subtalar joint. Accordingly, the sub-step 791 may include placing/securing/positioning such a trial implant based on the one or more significant points identified in the step 750. The sub-step 791 may also include the implementation of one or more instruments to facilitate placement and/or removal of the trial implant. In some aspects, one or more trial implants may be provided as components of a kit, system, or set configured to include various components to be implemented in conjunction with the performance of a subtalar arthroplasty procedure. In some aspects, the trial implant may be one of multiple trial implants provided as a set, with each of the trial implants having a different size. The trial implant as referenced in the sub-step 791 may also include a trial implant that consists of one or more components. The trial implant of the sub-step 791 may be a component configured to have the same geometry and other spatial geometry as one or more components of an implant to be placed and fixated in a subtalar arthroplasty procedure. The sub-step 791 may include placing the trial implant at least partially within the prepared joint space. That is to say that in some aspects one or more components of an implant may extend (e.g., laterally) beyond the dimensions/footprint of the calcaneum 100 and/or the talus 200. Accordingly, the implant trial may occupy a similar footprint, volume, and dimensions to those of one or more implants and/or components thereof (e.g., the implant of the disclosures referenced previously and incorporated herein in their entireties by reference). In some aspects, the sub-step 791 may include assessing one or more possible corrected positions of one or more structures of the subtalar joint once the trial implant is placed.

The step 790 is shown to include a sub-step 792 analyzing the fit of the trial implant within the prepared joint space, according to an exemplary method shown and described in FIG. 9. The sub-step 792 may include the implementation of one or more imaging, placement/preoperative planning technologies, or robotic/automated surgical technologies including, but not limited to, those described previously herein with reference to other sub-steps of the process 700. The fit (e.g., position, orientation, sizing, etc.) of the trial implant may be analyzed by one or more physicians or medical professionals through various means. For example, the trial implant may have measurements taken with respect to surrounding anatomical features, for example soft tissue structures, the calcaneum 100, the talus 200, and features thereof (such as those shown and described previously herein). Accordingly, one or more instruments may be implemented in sub-step 792 so as to facilitate the analysis and evaluation of the fit of the trial implant within the prepared joint space. In some aspects, such instruments may be provided as components of a kit such as that described previously herein. The sub-step 792 may also include analysis and/or testing/balancing of soft tissue structures of the areas adjacent to the subtalar joint while the trial occupies at least a portion of the joint space.

The step 790 is shown to include a sub-step 793 determining if the fit of the trial implant within the joint space is satisfactory, according to an exemplary method shown and described in FIG. 9. In some aspects, the sub-step 793 may include various criteria (e.g., measurements, imaging data, etc.) that are incorporated in the determination of whether the trial implant fit is satisfactory. Such a determination may also depend on the soft tissue analysis and balancing of the sub-step 793, for example. The sub-step 793 shown in FIG. 9 to have one of two possible outcomes each time it occurs. In some aspects, the sub-step 793 results in a determination that “yes” the fit of the trial implant within the joint space is satisfactory. In some aspects, the sub-step 793 results in a determination that “no” the fit of the trial implant is not satisfactory. The determination “no” results in a removal of the trial implant from the joint space and return to the first sub-step of the step 790, sub-step 791. Each time sub-step 791 is repeated, a trial implant of a different size/volume/dimension (or at least a portion thereof) than that which was most recently removed from the joint space is placed within the joint space and the step 790 is repeated beginning with the sub-step 791 until the determination “yes” is made. In some aspects, a determination of “no” may result to a return to the beginning of the process 700 and/or the step 710 for additional preparation.

The step 790 is shown to include a sub-step 794 confirming implant size based on the trial implant, according to an exemplary method shown and described in FIG. 9. The sub-step 794 directly follows the determination that “yes”, the fit of the trial implant within the joint space is satisfactory. Upon such a determination, the sub-step 794 is performed in order to identify a size of an implant and/or one or more components thereof that corresponds to that of the trial implant that provided the satisfactory fit within the joint space. In some aspects, measurements and/or imaging data that was applied to the decision of the sub-step 793 may be further analyzed in order to determine an implant size that will provide an implant fit consistent with the satisfactory trial implant fit.

The step 790 is shown to include a sub-step 795 selecting an implant with a size corresponding to that of the trial implant, according to an exemplary method shown and described in FIG. 9. Following the selection of an implant size in the sub-step 795, an implant is selected based on the sizing determined in the sub-step 794. In some aspects, this may include identifying an implant of a single size, while in other aspects this may include identifying multiple components (e.g., a calcaneal and a talar) of an implant that are available in one or more different sizes (which in some aspects may be compatible with various sizes of one another). Following the selection of the implant of the sub-step 795, the step 790 has been completed. However, as mentioned previously, the step 790 may include one or more sub-steps that may be repeated and/or be performed out of order. That is to say that if an implant size was selected but a physician was not confident said implant size corresponded to the trial implant that resulted in the determination “yes”, one or more sub-steps of the step 790 may be repeated.

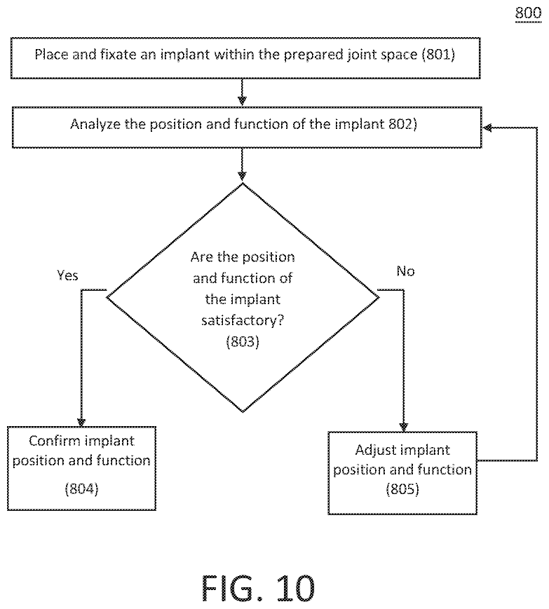

The process 700 is shown to include a step 800 placing and fixating (e.g., securing, using hardware, biologics, and/or other components, compositions, and/or techniques) an implant within the prepared joint space, according to the exemplary method shown and described in FIGS. 7-11. The step 800 may be performed one or more times and, in some aspects, may be an iterative process based on feedback received by the physician or other medical professional performing the step 800. Accordingly, the step 800 is shown to include various exemplary sub-steps of placing and fixating an implant withing the prepared joint space, which is shown and described with reference to FIG. 10. As with the step 800, the sub-steps of step 800 may also be repeated one or more times, in part or in full. As with the process 700, one or more steps of the process may be repeated, skipped, performed in an alternate order, or may be substituted for another step not shown in the exemplary embodiment of the sub-steps of FIG. 10.

The step 800 is shown to include a sub-step 801 placing and fixating an implant within the prepared joint space, according to an exemplary method shown and described in FIG. 10. In some aspects, the sub-step 801 may include implementing one or more means to secure one or more implant components. For example, the sub-step 801 may include implementing screws or other hardware configured to couple one or more components of the implant with the calcaneum 100 (e.g., such that the implant is adjacent/interfaces with the top surface 104) and/or the talus 200 (e.g., such that the implant is adjacent the bottom surface 204). In some aspects, a biologic solution such as a bone cement or similar coupling aid/adhesive agent may be implemented in conjunction with the aforementioned hardware. The sub-step 801 may include securing one or more components to the aforementioned structures, for example a first component coupled/secured with the calcaneum 100 and a second component coupled/secured with the talus 200. In some aspects, the sub-step 801 may include placing/securing/positioned one or more components of the implant relative to the one or more significant points identified in the step 750. For example, the sub-step 801 may include placing an implant with one or more centers of rotation, pivot points, or other points that correspond to a mechanism configured to mimic motion of a healthy subtalar joint. In some aspects, one or more components of the implant may include a porous or other similar structure configured to promote bone ingrowth and/or ongrowth. Accordingly, the sub-step 801 may include placing/securing/positioning such an implant based on the one or more significant points identified in the step 750. Further, in some aspects the sub-step 801 may include securing a third implant component, for example an intermediate component disposed between the calcaneum 100 and the talus 200 and in contact/interfacing with a calcaneal implant component and a talar implant component. In some aspects, the sub-step 801 may include the implementation of one or more instruments (some or all of which may be provided in a kit or system with the trial implants, the implant(s), and/or other components). For example, a first instrument may be implemented in securing a first implant component, while a second tool is subsequently implemented in securing a second implant component. In some aspects, the implant may be provided as a single component (e.g., already assembled or integral).

The step 800 is shown to include a sub-step 802 analyzing the position and function of the implant, according to an exemplary method shown and described in FIG. 10. The sub-step 802 may include the implementation of one or more imaging technologies the same as or similar to others described previously herein. For example, following implantation/fixation/securing of the implant with the calcaneum 100 and/or the talus 200, one or more physicians may analyze the position of the implant relative to various anatomical features (e.g., those shown and described previously, for example with reference to FIGS. 1-2). In some aspects, the sub-step 802 may include data collection, data analysis, and anatomic measurements incorporating the implant. Further, the sub-step 802 may also include the manipulation of the implant/joint (in some aspects, while using imaging technology) through normal subtalar range of motion (e.g., inversion and eversion) to evaluate if the implant meets various parameters, for example range of motion to a specified degree range (which may also include the implementation of one or more robotic/automated surgical technologies).

The step 800 is shown to include a sub-step 803 determining if the position and function of the implant are satisfactory, according to an exemplary method shown and described in FIG. 10. The sub-step 803 may be dependent on data, manipulation, and other outcomes of the sub-step 802. For example, if the implant is measured to be in the correct place relative to anatomic features/landmarks and/or the one or more estimated/determined significant points, a physician may deem that the position and function of the implant are satisfactory. A physician may also account for the range of motion when manipulating the joint and/or implant. However, if the implant is measured to be incorrectly placed (for example, does not meet the criteria mentioned above), a physician may elect to perform further testing or collect additional data, for example.

The step 800 is shown to include a sub-step 804 determination of “yes” the position and function of the implant are satisfactory, or a sub-step 805 determination of “no” the position and/or the function of the implant is/are not satisfactory, according to an exemplary method shown and described in FIG. 10. In the event that the determination “yes” is made, and the position and function of the implant are determined to be satisfactory, the step 800 is complete with the implant position and function confirmed. However, in the event that the determination “no” is made and the position and/or function of the implant is/are not determined to be satisfactory, the position and function of the implant may be adjusted and/or sub-step 801 may be repeated. In some aspects, this may include repositioning of the implant and/or other hardware. The step 800 may then be repeated until the sub-step 803 is completed and the determination “yes” is reached. However, as mentioned previously, the step 800 may include one or more sub-steps that may be repeated and/or be performed out of order. That is to say that if the determination “yes” was made and a physician subsequently questions the position and/or function of the implant, one or more sub-steps of the step 800 may be repeated. In some aspects, a determination of “no” may result to a return to the beginning of the process 700 and/or the sub-step 801 for additional preparation.

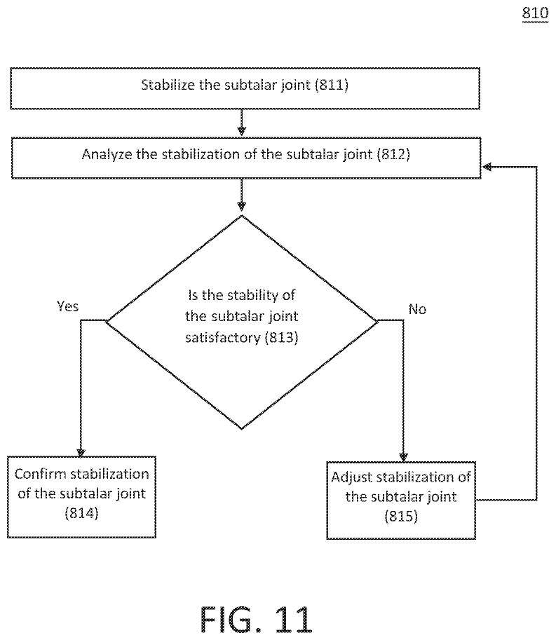

The process 700 is shown to include a step 810 stabilizing the subtalar joint, according to the exemplary method shown and described in FIGS. 7-11. The step 810 may be performed one or more times and, in some aspects may be an iterative process based on feedback received by the physician or other medical professional performing the step 820. Accordingly, the step 810 is shown various sub-steps of stabilizing the subtalar joint, which are shown and described with reference to FIG. 11. As with the step 810, the sub-steps thereof may also be repeated one or more times, in part or in full. As with the process 700, one or more sub-steps of the step 810 may be repeated, skipped, performed in an alternate order, or may be substituted for another step not shown in the exemplary embodiment of the step 810 of FIG. 11.

The step 810 is shown to include a sub-step 811 stabilizing the subtalar joint, according to the exemplary method shown and described in FIG. 11. The sub-step 811 may include incorporating one or more imaging technologies including but not limited to those shown and described previously in order to collect data directed to stability of the subtalar joint. In some aspects, the stability of the subtalar joint may be largely dependent on various positioning, tensioning, and balancing of forces present in various soft tissue structures adjacent the subtalar joint (e.g., ligaments, tendons, muscles, etc.). In some aspects, the sub-step 811 may include implementing one or more instruments in order to manipulate one or more soft tissue structures and/or measure forces present in the aforementioned soft tissue structures. For example, one or more instruments may be provided in conjunction with the implant and other components with said one or more instruments configured to be implemented in order to manipulate (e.g., reposition, shift, etc.) soft tissue. In some aspects, the sub-step 811 may also include balancing of soft tissues to address deformities, trauma, or degeneration present at or near the subtalar joint.

The step 810 is shown to include a sub-step 812 analyzing the stabilization of the subtalar joint, according to the exemplary method shown and described in FIG. 11. The sub-step 812 may include the implementation of imaging technology the same as and/or similar to that mentioned with respect to the sub-step 811 in order to analyze, measure, and collect data indicative of one or more forces or positions of the soft tissue structures adjacent the subtalar joint. In some aspects, instruments provided in a kit or system with the implant may be implemented in order to facilitate the analysis of the stability of the subtalar joint. Further, analyzing the stability of the subtalar joint may include manipulation of the subtalar joint (including the implant) so as to measure and observe behavior of soft tissue structures with respect to movement of the implant. In some aspects, the physician may have criteria (e.g., forces, positions, range of motion, etc.) which the various soft tissue structures must satisfy.

The step 810 is shown to include a sub-step 813, determining if the stability of the subtalar joint is satisfactory, according to the exemplary method shown and described in FIG. 11. The sub-step 813 may be dependent on data, manipulation, and other outcomes of the sub-step 812. For example, if the stability of the subtalar joint (e.g., soft tissue structures) is measured to meet criteria relative to anatomic features/landmarks and/or the one or more estimated/determined significant points, a physician may deem that the stability of the subtalar joint is satisfactory. A physician may also account for range of motion when manipulating the joint and surrounding soft tissue structures. However, if the subtalar joint is measured to be unstable and/or incorrectly balanced (for example, does not meet the criteria mentioned above), a physician may elect to perform further testing or collect additional data and/or repeat necessary sub-steps to establish and confirm stability.

The step 810 is shown to include a sub-step 813 determination of “yes” the stabilization of the subtalar joint is satisfactory, or a sub-step 814 determination of “no” the stabilization of the subtalar joint is not satisfactory, according to an exemplary method shown and described in FIG. 11. In the event that the determination “yes” is made, and the stabilization of the subtalar joint is determined to be satisfactory, the step 800 is complete with the stabilization of the subtalar joint confirmed. However, in the event that the determination “no” is made and the stabilization of the subtalar joint is not determined to be satisfactory, the stabilization of the subtalar joint be adjusted and/or sub-step 801 may be repeated. In some aspects, this may include manipulation of one or more of the soft tissue structures relative to the subtalar joint/implant and/or the calcaneum 100 and/or the talus 200. The step 800 may then be repeated until the sub-step 813 is completed and the determination “yes” is reached. However, as mentioned previously the step 800 may include one or more sub-steps that may be repeated and/or be performed out of order. That is to say that if the determination “yes” was made and a physician subsequently questions the stability of the subtalar joint, one or more sub-steps of the step 800 may be repeated (and/or various steps of the process 700). In some aspects, a determination of “no” may result to a return to the beginning of the process 700 and/or the step 710 for additional preparation.

The process 700 is shown to include a step 820 restoring any necessary soft tissue structures and repairing the fibula, according to the exemplary method shown and described in FIGS. 7-11. The step 820, in some aspects, is dependent on the steps 710, 720, and 730. Depending on where the at least one incision was made and the soft tissue structures that were encountered/contacted in steps 720 and 730, similar and/or the same soft tissue structures may require restoration after the subtalar implant has been placed. For example, if the one or more incisions made included a lateral incision, a physician may restore one or more of the calcaneofibular ligament, the anterior talofibular ligament, and/or the extensor digitorium brevis. Similarly, if the one or more incisions made included a posterior incision, a physician may repair at least a portion of the Achilles and/or other tendons. Additionally, the step 820 includes repairing the fibular osteotomy that was performed in the sub-steps of the step 710 shown and described with reference to FIG. 8. In some aspects, a physician may implement various plates, fasteners, wedges, or other hardware/implants in performing said repair. Further, in some aspects the physician may reference preoperative and/or intraoperative imaging in performing the fibular repair of the step 820.

The process 700, as well as the various sub-steps included therein (and referred to according to steps 710, 790, 800, and 810) may also be performed/implemented in conjunction with a patient-specific implant (PSI) as well as other components of a PSI system (e.g., imaging technology, software, jigs, cut guides, instrumentation, etc.). For example, one or more sets of imaging data (e.g., collected using one or more of the imaging technologies mentioned herein) may be used to collect patient data prior to a subtalar arthroplasty procedure (e.g., the process 700). This data may then be used to create (e.g., 3-D print, machine, etc.) one or more PSI components such as those mentioned previously. For example, one or more components of a subtalar joint implant may include a geometry that is based on the patient data collected prior to the subtalar arthroplasty procedure. Similarly, the step 770 of making one or more primary cuts in at least the calcaneum or the talus may include the use of one or more PSI cut guides and/or jigs. Such cut guides/jigs may be created based on the patient data collected and configured to guide one or more cuts so as to access and/or avoid various anatomical features. It should also be noted that PSI instrumentation may also be implemented in one or more of the steps of the process 700.

The process 700 is shown to include a step 830, closing the at least one incision, according to the exemplary method shown and described in FIGS. 7-11. In some aspects, the step 830 may include closing one or more incisions using sutures or other fixation members and/or adhesives. Antibacterial agents may also be introduced at or near the incision in the step 830. The step 830 is dependent upon the at least one incision made in the step 710. In some aspects, only one incision was made in the step 710 and, thus, only one incision requires closing in the step 830 However, in other aspects multiple incisions may be made in the step 710 and accordingly multiple incisions require closing in the step 830.

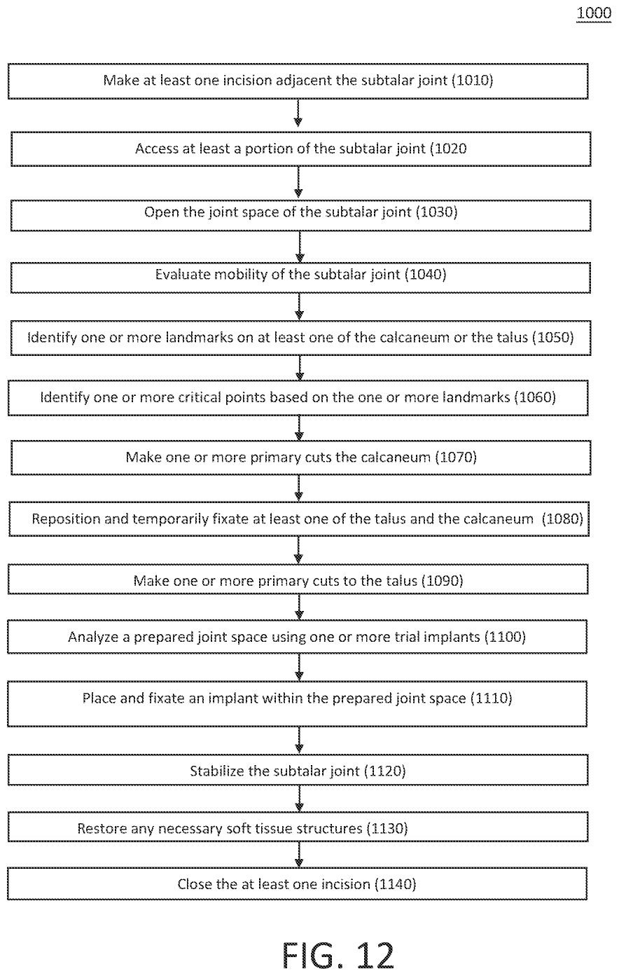

Referring now to FIGS. 12-16, a second exemplary process 1000 for performing a subtalar arthroplasty procedure, according to some aspects is described. Similar to the process 700 shown and described with reference to FIGS. 7-11, the process 700 includes various steps that include one or more sub-steps. Subtalar arthroplasty, as with all medical procedures, should be performed by one or more physicians, surgeons, or otherwise adequately trained healthcare providers and is shown and described as such. It should be understood that the steps of the process 1000 (as well as the steps of the associated sub-processes) may be skipped, performed in an alternate order, repeated, or otherwise modified in performing a subtalar arthroplasty. In some aspects, alternate steps may be added and/or substituted for the steps shown and described herein with reference to the process 1000. Furthermore, it should also be understood that the steps of the process 1000 may not be limiting; that is to say that in some aspects, additional steps may be performed in order to perform a subtalar arthroplasty. The steps of process 1000 may be performed with one or more of the implants, instruments, devices, or other concepts discussed in the issued patents and pending patent applications discussed previously and incorporated by reference herein in their entireties. Further, in some aspects, one or more implants and/or pieces thereof may be implanted according to the process 1000, for example an implant may include multiple pieces configured to interface with or adjacent to the posterior facet 106, the middle facet 108, the anterior facet 110, or other anatomical features. In some aspects, additional and/or alternative implants, instruments, devices, and/or other concepts to those incorporated by reference herein in their entireties may be implemented in conjunction with that that is shown and described herein.

Referring now to FIG. 12, the process 1000 is shown to include a step 1010 making at least one incision substantially adjacent to a subtalar joint, according to the exemplary method shown and described in FIGS. 7-11. In some aspects, the at least one incision may be an incision extending from the adjacent the distal tip of the fibula extending to/along a trajectory toward the middle of the cuboid of the fourth ray such that the subtalar joint may be accessed via an anteriorly, laterally, and/or anterior-laterally approach (e.g., thus facilitating a sinus tarsi approach). However, in some aspects, one or more circumstances (e.g., trauma, deformity, previous procedures, minimize surgical morbidity, etc.) may be present that are conducive to alternate incisions and/or directional approaches. For example, in some aspects, the at least one incision may include an extended lateral incision/approach (e.g., in which a vertical limb meets a horizontal limb and the soft tissues on the side of the calcaneum are lifted off to access the lateral wall of the calcaneum and the subtalar joint). Additional incisions may also be made at, near, or adjacent to the subtalar joint in order to provide optimal visualization of the subtalar joint and surrounding soft tissue structures. In some aspects, a subtalar arthroplasty may include removal of metalwork, for example after trauma where previous metalwork is in the calcaneum and/or talus and requires removal during a subtalar arthroplasty procedure, and accordingly the step 1010 may include making one or more incisions configured to facilitate the removal of said metalwork. Further, in some aspects, other alternate incisions and/or approaches may be taken, for example a posterior-lateral approach and/or a posterior approach (which, in some aspects, may require manipulating/taking down/severing the Achilles tendon). In some aspects, incisions may be made that are not directly adjacent to the joint. Additionally, in some aspects, a patient may have one or more conditions or circumstances that warrant a medial approach, for example trauma has occurred to one or more portions of the foot, ankle, and/or lower leg that prohibit approaches/incisions in directions other than the medial direction relative to the subtalar joint. Accordingly, in such circumstances a medial incision may be made. It should be understood that the step 1010 may also include multiple incisions being made which may include a combination of two or more of the aforementioned incisions and/or alternate incisions (e.g., a posterior and a medial incision, a medial and a lateral incision, etc.).

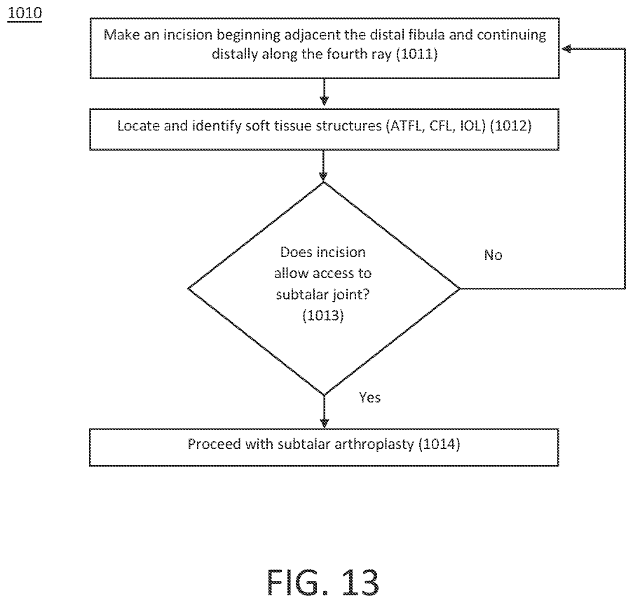

The step 1010 is shown to include an exemplary sub-process as shown in FIG. 13 which, according to some aspects, includes sub-steps which may be performed in performing the step 1010. It should be understood that one or multiple sub-steps of the step 1010 may be omitted, repeated, or performed in an alternate order to that shown in the sub-process of FIG. 13. FIG. 13 is shown to include a sub-step 1011 of making an incision adjacent the distal fibula and continuing distally to the middle of the cuboid of the fourth ray, according to an exemplary method. In some aspects, the incision of sub-step 1011 may overlap (e.g., an additional incision is not made in sub-step 1011) with the step 1010 in that a single incision may be made so long as the single incision suffices to provide necessary access to the subtalar joint and adjacent structures. In some aspects, the incision of the step 1011 may be extended or otherwise modified based on an evaluation of the access to the subtalar joint and adjacent structures following the making of the incision.

The step 1010 is further shown to include a sub-step 1012 of locating and identifying soft tissue structures, according to some aspects. The sub-step 1012 may include the identification of one or more soft tissue structures including but not limited to the ATFL, the CFL, and the IOL. In some aspects, the sub-step 1012 may include repositioning one or more soft tissue structures adjacent the subtalar joint so as to achieve access to the joint. Further, the sub-step 1012 may also include severing, resecting, or otherwise modifying one or more soft tissue structures in the event that one or more of said soft tissue structures obstructs access to the subtalar joint and adjacent structure and a determination that such structure(s) cannot be repositioned. In some aspects, the sub-step 1012 may include the removal of various soft tissue elements (e.g., structures or otherwise) including but not limited to those identified previously herein. Additionally, the sub-step 1012 may include various blunt dissection or other manipulation of soft tissue structures (by hand or using instruments).

The step 1010 is further shown to include a sub-step 1013 of evaluating access to the subtalar joint, according to some aspects. After making the incision and identifying/manipulating any necessary soft tissue in sub-steps 1011, 1012, the physician may evaluate the access to the subtalar joint. In some aspects, the sub-step 1013 may include the physician manipulating the subtalar joint, ankle joint, and/or other anatomical structures in order to evaluate access to the subtalar joint. In the event that the physician determines that access to the subtalar joint is insufficient, the physician may repeat the sub-steps 1011, 1012 until a determination is made that there is sufficient access to the subtalar joint. Once such a determination is made, the physician may proceed to a sub-step 1014 of proceeding with the subtalar arthroplasty procedure. In some aspects, a physician may implement a fibular osteotomy such as that shown and described with reference to the step 710 and sub-steps thereof, for example in the event that a determination is made that sufficient access to the subtalar joint cannot be achieved otherwise.

Referring again to FIG. 12, the process 1000 is shown to include a step 1020 of accessing at least a portion of the subtalar joint, according to the exemplary method shown and described in FIGS. 12-16. The step 1020 may be performed directly after the step 1010 as shown and described previously, for example at least a portion of the subtalar joint may be accessed via the at least one incision made in the step 1010. Further, the step 1020 may in some aspects be dependent on the one or more incisions made in the step 1010 (e.g., if a lateral incision was made in the step 1010, then the step 1020 may include accessing the subtalar joint from an anterior-lateral direction). It should also be noted that the step 1020 may include the manipulation, repositioning, and/or resection of one or more soft tissue structures disposed between the one or more incision made in the step 1010. For example, if a lateral incision was made in the step 1010, the step 1020 may include manipulating, repositioning, bluntly dissecting, etc. one or more of the calcaneofibular ligament, the anterior talofibular ligament, and/or the extensor digitorium brevis (and/or other soft tissue structures which may have been previously identified and/or preliminarily manipulated in sub-steps of the step 1010 as shown and described previously herein). The soft tissue structures that may be contacted or otherwise addressed in the step 1010 (including sub-steps thereof) may be dependent on the one or more incisions made in the step 1010.

The process 1000 is shown to include a step 1030 opening a joint space of the subtalar joint, according to the exemplary method shown and described in FIGS. 12-16. The step 1030 may include implementing one or more instruments common to arthroplasty procedures including, for example laminar spreaders, bone distractors over wires, elevators, osteotomes, sensors for ligament balancing, tracking arrays for bony alignment, PSI components, etc. Similar to the step 1020, the step 1030 may be dependent on the one or more incisions made in the step 1010. For example, the instruments and approach implemented in the opening the joint space may be dependent on whether the joint is being accessed from a lateral, posterior lateral, posterior, medial, or other directional approaches. Further, the step 1030 and/or the instruments implemented in conjunction 1030 may be dependent on the geometric features of the subtalar joint and components thereof (e.g., the calcaneum and the talus). For example, in accessing the joint laterally, the physician may implement an instrument that is conducive to contacting the talus at or adjacent the sinus tarsi (disposed on the talus, but typically positioned adjacent the middle facet). Conversely, in accessing the subtalar joint posteriorly, the physician may implement one or more instruments conducive to contacting the posterior facet as shown and described with reference to FIG. 1.

The process 1000 is shown to include a step 1040 evaluating mobility of the subtalar joint, according to the exemplary method shown and described in FIGS. 12-16. The step 1040 may include the physician immobilizing and/or manipulating various anatomical structures including the subtalar joint as well as other structures adjacent the subtalar joint (e.g., tibia ankle joint, soft tissue structures, etc.). In some aspects, the step 1040 may include the physician performing various range of motion (ROM) testing and measuring the ROM of the subtalar joint. In some aspects, the step 1040 may result in a determination that one or more of the preceding steps of the process 1000 need to be repeated, for example if the access to the subtalar joint is insufficient relative to the mobility of the subtalar joint that is evaluated in the step 1040. In evaluating the ROM of the subtalar joint, the physician may record various measurements against which mobility of the subtalar joint may be compared later in the procedure/process 1000 (e.g., after arthroplasty has been performed).

The process 1000 is shown to include a step 1050 identifying one or more landmarks on at least one of the calcaneum or the talus, according to the exemplary method shown and described in FIGS. 12-16. The step 1050 may include identifying one or more of the geometric features of the calcaneum 100 and/or the talus 200 shown and described with reference to FIGS. 1-6, for example the posterior facet 106, the middle facet 108, the anterior facet 110, the talar head 206, the talar neck 208, etc. In some aspects, the step 1050 may be dependent on one or more of the previous steps of the process 1000 in that the landmarks that may be identifiable on the calcaneum 100 and/or the talus 200 may be dependent on the directional approach taken by a physician (e.g., dependent on the one or more incisions made and whether said incisions were made laterally, posterior-laterally, posterior, medial, etc. relative to the subtalar joint). In some aspects, the step 1050 (and subsequent step 1060) may further include the implementation of one or more imaging methods. For example, fluoroscope, CT, MRI, X-ray, or other imaging technologies may be implemented in order to capture images of the calcaneum 100, the talus 200 (and/or portions thereof), and/or anatomical components adjacent the subtalar joint for analysis by one or more physicians. As mentioned previously, in some aspects, one or more components of the calcaneum 100, the talus 200, or other anatomical features adjacent the subtalar joint may not be present or may not be identifiable (e.g., trauma, degeneration, deformity, etc.). In some aspects, the step 1050 may include identifying one or more of the aforementioned landmarks with respect to one or more of the other aforementioned landmarks (or other anatomical landmarks entirely). For example, if the middle facet 108 and the anterior facet 110 abut one another, the posterior facet 108 may be identified as a landmark and furthermore may be identified (e.g., measured, mapped, etc.) with respect to landmarks of the calcaneum 100 other than the middle facet 108 and the anterior facet 110 or with respect to landmarks of the talus 200, for example the talar head 206. The step 1050 may also include the identification and measurement/estimation of one or more angles associated with the calcaneum 100, the talus 200, the subtalar joint, or other anatomical features/structures, for example the angle of Gissane. Soft tissue landmarks may also be identified including the soft tissue structures mentioned previously as well other soft tissue structures (which may depend on directional approach) for example the interosseous ligament, the deltoid ligament, peroneal tendons, calcaneo-fibular ligament, sural nerve, superficial peroneal nerve, neurovascular bundle (if medial approach), etc.

The process 1000 is shown to include a step 1060 estimating a significant point of the subtalar joint based on the one or more landmarks, according to the exemplary method shown and described in FIGS. 12-16. In some aspects, the step 1060 may include estimating (or, in some aspects, determining) one or more significant points of the subtalar joint based on the one or more landmarks. The step 1060 may further include the application of one or more algorithms, software programs, mathematical models, or other tools in order to analyze the landmarks identified (and in some cases imaged) in the step 1050. Additionally, significant points of the subtalar joint may include and/or be referenced/identified with respect to one or more of the geometric features as shown and described previously with reference to FIGS. 1-6 (e.g., talar head, talar neck, one or more facets of the calcaneum, etc.). Further, other anatomical structures that may be positioned adjacent the subtalar joint (or in some research considered to be a part of the subtalar joint, for example the navicular) may also be incorporated in the identification of the one or more significant points. The significant points of the step 1060 may include one or more centers of rotation, axis/axes, articulation points, pivot points (e.g., multiple pivot points along a line), one or more points along or central to a spline (e.g., piecewise polynomial function), ellipse, or other geometric curve or shape. Further, the one or more significant points of the step 1060 may correspond to the aforementioned motion (e.g., inversion and eversion) of the subtalar joint. For example, the one or more significant points may correspond with one or more kinematic/mechanical properties and/or movements of the subtalar joint and, accordingly, correspond to one or more components of an implant to be implanted in a subtalar arthroplasty procedure. In some aspects, the one or more significant points may further correspond to one or more sizes of one or more components of an implant to be placed and fixated in the total arthroplasty procedure included in the process 1000. For example, the location of one or more significant points (e.g., centers of rotation) estimated/determined/located on the top surface 104 of the calcaneum 100 may correspond to a size and/or placement/fixation location of one or more implant components. Similarly, one or more estimated/determined significant points and the location/proximity of said one or more significant points to one or more of the anatomic landmarks as shown and described previously may also correspond to sizing, placement, and/or selection of one or more components of an implant.

The steps 1050 and 1060 may also include the identification of additional landmarks which may serve as a basis for one or more cut/resection planes for the calcaneum and/or the talus. For example, in some aspects, the steps 1050, 1060 may include the identification of the lateral sulcus of the calcaneum and the sinus tarsi of the talus (e.g., point “A”). The steps 1050, 1060 may further include identifying a range (e.g., (e.g., a radius of 2-5 mm deep from the top surface of the middle facet 108) from a point on the anterior portion of middle facet 108 (e.g., the middle, edge, etc. of the middle facet 108) such that a cut within said range may remove 2-3 mm from the middle facet 108 (e.g., point “B”). Further, a point may be identified on the middle facet 108 where a radius is identified extending in the inferior direction to identify a point that serves as a reference point and/or establishes at least a portion of a plane in conjunction with other identified points/landmarks (e.g., lateral sulcus and/or sinus tarsi). In some aspects, an additional landmark/point may be identified based on a point on a lateral apex of the posterior face 106. From said lateral apex, a plane may be identified that intersects with a sphere having a radius (e.g., 5-7 mm, 3-9 mm, etc.). In alternate aspects, an additional landmark/point may be identified by creating a line with point “A” and point “B” as defined above such that said line is substantially in the coronal plane of the subtalar joint (not necessarily the physiological coronal plane) based on positioning of the talus. In some aspects, a cut plane may be rotated about said line from point “A” to point “B” where said cut plane is approximately between 5 and 40 degrees above the horizontal plane. Additionally, one or more points/lines may be identified to as to establish one or more additional boundaries for the aforementioned cut plane. In some aspects, a point may be located by pivoting about point “A” so as to identify a point positioned between the middle facet 108 and the anterior facet 110 which may, in some aspects, be between 5 and 25 degrees (e.g., 10 degrees) in a clockwise direction from point “B” as identified above. In some aspects, the process 1000 may include creating one or more cut guides or instruments based on the points/landmarks/lines/angles described with reference to the steps 1050, 1060, where said guides are coupled with the anatomy of or adjacent to the subtalar joint in subsequent steps of the process 1000.

The process 1000 is shown to include a step 1070 making one or more primary cuts in the calcaneum, according to the exemplary method shown and described in FIGS. 12-16. In some aspects, the step 1070 may include making one or more cuts according to the points (e.g., “A” and “B”) as located, identified, and established in the steps 1050, 1060. Further, in some aspects, the step 1070 may include making one or more resection cuts along one or more cut lines as described with reference to the steps 1050, 1060. As mentioned previously with reference to prior steps in the process 1000, the step 1070 may depend at least in part on the one or more incisions made in the step 1010. For example, if the subtalar joint is accessed laterally, step 1070 may include preparing (e.g., at least making one or more cuts to) the lateral-most portions of one or more surfaces (e.g., articular, interfacing, etc.) of the calcaneum prior to preparing the medial-most portions. With reference to FIGS. 1-2, the top surface 104 and the bottom surface 204 of the calcaneum 100 and the talus 200, respectively, typically interface with cartilage and a subtalar arthroplasty procedure typically includes removing at least some (or in some aspects, all) of said cartilage and/or subchondral bone from the aforementioned surfaces. In some aspects, the step 1080 may include the removal of the aforementioned cartilage such that the top surface 104 of the calcaneum 100 and the bottom surface 204 of the talus 200 (which may be further resected and/or otherwise cut in subsequent steps) may contact the majority of a subtalar implant that is implanted and fixated between the calcaneum 100 and the talus 200 (and thus interfacing with the top surface 104 and the bottom surface 204). In some aspects, the step 1070 may further include implementing one or more instruments in order to modify the top surface 104 of the calcaneum 100. For example, in some aspects, a physician may score (e.g., create an uneven texture by creating one or more depressions in the top surface 104) so as to promote fixation of and/or further preparation of the top surface 104 for arthroplasty.

The process 1000 is shown to include a step 1090 repositioning and temporarily fixating at least one of the calcaneum or the talus. In some aspects, the calcaneum and talus may both be fixed (e.g., with temporary fixation elements, k-wires, stabilization wires, etc.) to one another and/or to adjacent anatomy prior to any cuts being made (e.g., the cut(s) of step 1070 may, in some aspects, be performed after the step 1090 is performed). In some aspects, only the talus may be temporarily fixed to one or more adjacent anatomic components prior to a talar cut/cuts being made. Further, in some aspects, the talus may be manipulated and/or repositioned prior to being temporarily fixated. For example, the physician may place one or more k-wires (or other wires mentioned previously which, for brevity's sake, will be considered interchangeable with k-wires) in the talus and internally rotate the talus (e.g., using the k-wire as a sort of handle or “joystick”, rotate counterclockwise from a superior view on the right foot/talus of a patient). In performing such rotation of the talus, the physician may expose space below an anterior portion of the talus near the anterior facet 110 and also manipulate (e.g., pull) an inferior articulating surface of the talus into the physician's view. Such manipulation may provide the physician better visibility of the lower talar articular surface and, accordingly, the talus may then be temporarily fixated in the previously described position either using k-wires and/or a specified instrument/jig (which may be a PSI component). In some aspects, a cut guide (e.g., PSI cut guide) or other instrument/component may be configured to fit the talus of the patient in the specific position described previously such that, if the guide or instrument does not fit, the physician is aware that the talus was either under or over-rotated.

The process 1000 is shown to include a step 1090 making one or more primary cuts in the talus, according to the exemplary method shown and described in FIGS. 12-16. In some aspects, the step 1090 may include making one or more cuts according to one or more PSI cut guides or instruments produced based on preoperative imaging. Further, in some aspects, the step 1090 may include making one or more resection cuts along one or more cut lines of the talus that have been exposed by the manipulation of the talus as described in the step 1080. As mentioned previously with reference to prior steps in the process 1000, the step 1090 may depend at least in part on the one or more incisions made in the step 1010. For example, if the subtalar joint is accessed laterally, step 1090 may include preparing (e.g., at least making one or more cuts to) the lateral-most portions of one or more surfaces (e.g., articular, interfacing, etc.) of the talus prior to preparing the medial-most portions. With reference to FIGS. 1-2, the top surface 104 and the bottom surface 204 of the calcaneum 100 and the talus 200, respectively, typically interface with cartilage and a subtalar arthroplasty procedure typically includes removing at least some (or in some aspects, all) of said cartilage and/or subchondral bone from the aforementioned surfaces. In some aspects, the step 1090 may include the removal of the aforementioned cartilage such that the top surface 104 of the calcaneum 100 and the bottom surface 204 of the talus 200 (which may be further resected and/or otherwise cut subsequently) may contact the majority of a subtalar implant that is implanted and fixated between the calcaneum 100 and the talus 200 (and thus interfacing with the top surface 104 and the bottom surface 204). In some aspects, the step 1090 may further include implementing one or more instruments in order to modify the bottom surface 204 of the talus 200. For example, in some aspects, a physician may score (e.g., create an uneven texture by creating one or more depressions in the top surface 104) so as to promote fixation of and/or further preparation of the bottom surface 204 for arthroplasty. In some aspects, the step 1090 may include the placement of one or more cut guides and/or jigs configured to guide one or more cuts, including but not limited to PSI cut guides.

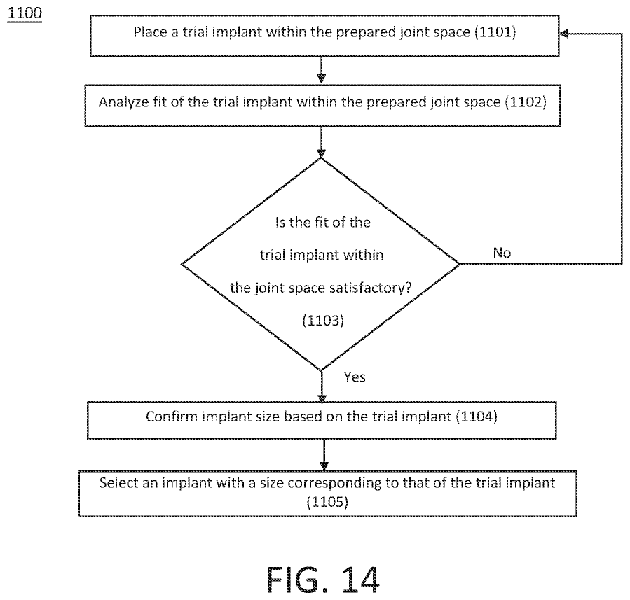

The process 1000 is shown to include a step 1100 analyzing a prepared joint space using one or more trial implants, according to the exemplary method shown and described in FIGS. 12-16. The step 1100 may be performed one or more times and, in some aspects, may be an iterative process based on feedback received by the physician or other medical professional performing the step 1100. Accordingly, the step 1100 is shown as a collection of sub-steps shown and described with reference to FIG. 14. The step 1100 may be repeated one or more times, in part or in full. As with the process 1000, one or more sub-steps of the step 1100 may be repeated, skipped, performed in an alternate order, or may be substituted for another step not shown in the exemplary sub-steps of the step 1100 of FIG. 14.

The step 1100 is shown to include a sub-step 1101 placing a trial implant within the prepared joint space, according to an exemplary method/sub-process shown and described in FIG. 14. The trial implant may be placed such that at least a first portion of the trial implant is in contact with the top surface 104 of the calcaneum and at least a second portion of the trial implant is in contact with the bottom surface 204 of the talus. It should be noted that in some aspects, trial implants may be placed both before and after cuts are made, surfaces are prepared, etc. In some aspects, the sub-step 1101 may include placing/securing/positioned one or more components of the trial implant relative to the one or more significant points identified in the step 1050. For example, the sub-step 1101 may include placing a trial implant with features that correspond to one or more centers of rotation, pivot points, or other points included on an implant that correspond to a mechanism configured to mimic motion of a healthy subtalar joint. Accordingly, the sub-step 1101 may include placing/securing/positioning such a trial implant based on the one or more significant points identified in the step 1050. The sub-step 1101 may also include the implementation of one or more instruments to facilitate placement and/or removal of the trial implant. In some aspects, one or more trial implants may be provided as components of a kit, system, or other set configured to include various components to be implemented in conjunction with the performance of a subtalar arthroplasty procedure. In some aspects, the trial implant may be one of multiple trial implants provided as a set, with each of the trial implants having a different size. The trial implant as referenced in the sub-step 1101 may also include a trial implant that consists of one or more components. The trial implant of the sub-step 1101 may be a component configured to have the same geometry and other spatial geometry as one or more components of an implant to be placed and fixated in a subtalar arthroplasty procedure. The sub-step 1101 may include placing the trial implant at least partially within the prepared joint space. That is to say that in some aspects, one or more components of an implant may extend (e.g., laterally) beyond the dimensions/footprint of the calcaneum 100 and/or the talus 200. Accordingly, the implant trial may occupy a similar footprint, volume, and dimensions to those of one or more implants and/or components thereof (e.g., the implant of the disclosures referenced previously and incorporated herein in their entireties by reference). In some aspects, the sub-step 1101 may include assessing one or more possible corrected positions of one or more structures of the subtalar joint once the trial implant is placed.

The step 1100 is shown to include a sub-step 1102 analyzing the fit of the trial implant within the prepared joint space, according to an exemplary method shown and described in FIG. 14. The sub-step 1102 may include the implementation of one or more imaging, placement/preoperative planning technologies, or robotic/automated surgical technologies including but not limited to those described previously herein with reference to other sub-steps of the process 1000. The fit (e.g., position, orientation, sizing, etc.) of the trial implant may be analyzed by one or more physicians or medical professionals through various means. For example, the trial implant may have measurements taken with respect to surrounding anatomical features, for example soft tissue structures, the calcaneum 100, the talus 200, and features thereof (such as those shown and described previously herein). Accordingly, one or more instruments may be implemented in sub-step 1102 so as to facilitate the analysis and evaluation of the fit of the trial implant within the prepared joint space. In some aspects, such instruments may be provided as components of a kit such as that described previously herein. The sub-step 1102 may also include analysis and/or testing/balancing of soft tissue structures of the areas adjacent to the subtalar joint while the trial occupies at least a portion of the joint space.

The step 1100 is shown to include a sub-step 1103 determining if the fit of the trial implant within the joint space is satisfactory, according to an exemplary method shown and described in FIG. 14. In some aspects, the sub-step 1103 may include various criteria (e.g., measurements, imaging data, etc.) that are incorporated in the determination of if the trial implant fit is satisfactory. Such a determination may also depend on the soft tissue analysis and balancing of sub-step 1103, for example. The sub-step 1103 shown in FIG. 14 to have one of two possible outcomes each time it occurs. In some aspects, the sub-step 1103 results in a determination that “yes” the fit of the trial implant within the joint space is satisfactory. In some aspects, the sub-step 1103 results in a determination that “no” the fit of the trial implant is not satisfactory. The determination “no” results in the removal of the trial implant from the joint space and return to the first sub-step of the step 1100, sub-step 1101. Each time sub-step 1101 is repeated, a trial implant of a different size/volume/dimension (or at least a portion thereof) than that which was most recently removed from the joint space is placed within the joint space and the step 1100 is repeated beginning with the sub-step 1101 until the determination “yes” is made. In some aspects, a determination of “no” may result to a return to the beginning of the process 1000 and/or the step 1010 for additional preparation.

The step 1100 is shown to include a sub-step 1104 confirming implant size based on the trial implant, according to an exemplary method shown and described in FIG. 14. The sub-step 1104 directly follows the determination that “yes”, the fit of the trial implant within the joint space is satisfactory. Upon such a determination, the sub-step 1104 is performed in order to identify a size of an implant and/or one or more components thereof that corresponds to that of the trial implant that provided the satisfactory fit within the joint space. In some aspects, measurements and/or imaging data that was applied to the decision of the sub-step 1103 may be further analyzed in order to determine an implant size that will provide an implant fit consistent with the satisfactory trial implant fit.

The step 1100 is shown to include a sub-step 1105 selecting an implant with a size corresponding to that of the trial implant, according to an exemplary method shown and described in FIG. 14. Following the selection of an implant size in the sub-step 1105, an implant is selected based on the sizing determined in the sub-step 1104. In some aspects, this may include identifying an implant of a single size, while in other aspects this may include identifying multiple components (e.g., a calcaneal and a talar) of an implant that are available in one or more different sizes (which in some aspects may be compatible with various sizes of one another). Following the selection of the implant of the sub-step 1105, the step 1100 has been completed. However, as mentioned previously the step 1100 may include one or more sub-steps that may be repeated and/or be performed out of order. That is to say that if an implant size was selected but a physician was not confident said implant size corresponded to the trial implant that resulted in the determination “yes”, one or more sub-steps of the step 1100 may be repeated.

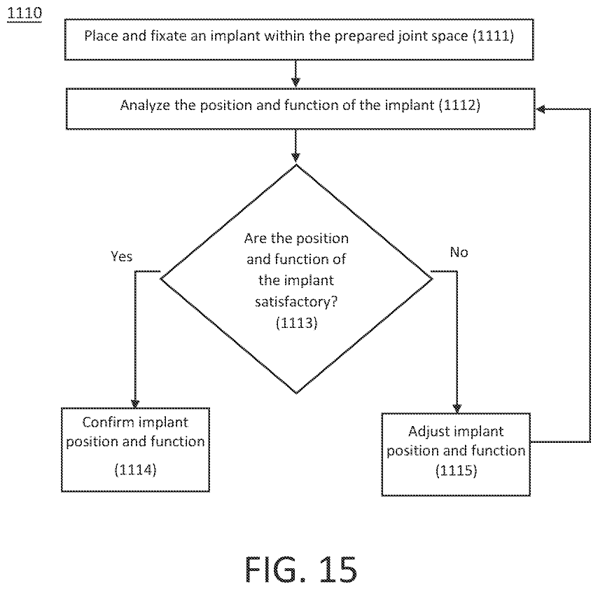

The process 1000 is shown to include a step 1110 (see FIG. 15) placing and fixating (e.g., securing, using hardware, biologics, and/or other components, compositions, and/or techniques) an implant within the prepared joint space, according to the exemplary method shown and described in FIGS. 12-16. The step 1110 may be performed one or more times and, in some aspects, may be an iterative process based on feedback received by the physician or other medical professional performing the step 1110. Accordingly, the step 1110 is shown to include various exemplary sub-steps of placing and fixating an implant withing the prepared joint space, which is shown and described with reference to FIG. 15. As with the step 1110, the sub-steps of step 1110 may also be repeated one or more times, in part or in full. As with the process 1000, one or more steps of the process may be repeated, skipped, performed in an alternate order, or may be substituted for another step not shown in the exemplary embodiment of the sub-steps of FIG. 15.

The step 1110 is shown to include a sub-step 1111 placing and fixating an implant within the prepared joint space, according to an exemplary method shown and described in FIG. 15. In some aspects, the sub-step 1111 may include implementing one or more means to secure one or more implant components. For example, the sub-step 1111 may include implementing screws or other hardware configured to couple one or more components of the implant with the calcaneum 100 (e.g., such that the implant is adjacent/interfaces with the top surface 104) and/or the talus 200 (e.g., such that the implant is adjacent the bottom surface 204). In some aspects, a biologic solution such as a bone cement or similar coupling aid/adhesive agent may be implemented in conjunction with the aforementioned hardware. The sub-step 1111 may include securing one or more components to the aforementioned structures, for example a first component coupled/secured with the calcaneum 100 and a second component coupled/secured with the talus 200. In some aspects, the sub-step 1111 may include placing/securing/positioned one or more components of the implant relative to the one or more significant points identified in the step 1050. For example, the sub-step 1111 may include placing an implant with one or more centers of rotation, pivot points, or other points that correspond to a mechanism configured to mimic motion of a healthy subtalar joint. In some aspects, one or more components of the implant may include a porous or other similar structure configured to promote bone ingrowth and/or ongrowth. Accordingly, the sub-step 1111 may include placing/securing/positioning such an implant based on the one or more significant points identified in the step 1050. Further, in some aspects the sub-step 1111 may include securing a third implant component, for example an intermediate component disposed between the calcaneum 100 and the talus 200 and in contact/interfacing with a calcaneal implant component and a talar implant component. In some aspects, the sub-step 1111 may include the implementation of one or more instruments (some or all of which may be provided in a set, kit or system with the trial implants, the implant (s), and/or other components). For example, a first instrument may be implemented in securing a first implant component, while a second tool is subsequently implemented in securing a second implant component. In some aspects, the implant may be provided as a single component (e.g., already assembled or integral).

The step 1110 is shown to include a sub-step 1112 analyzing the position and function of the implant, according to an exemplary method shown and described in FIG. 15. The sub-step 1112 may include the implementation of one or more imaging technologies the same as or similar to others described previously herein. For example, following implantation/fixation/securing of the implant with the calcaneum 100 and/or the talus 200, one or more physicians may analyze the position of the implant relative to various anatomical features (e.g., those shown and described previously, for example with reference to FIGS. 1-2). In some aspects, the sub-step 1112 may include data collection, data analysis, and anatomic measurements incorporating the implant. Further, the sub-step 1112 may also include the manipulation of the implant/joint (in some aspects, while using imaging technology) through normal subtalar ROM (e.g., inversion and eversion) to evaluate if the implant meets various parameters, for example range of motion to a specified degree range (which may also include the implementation of one or more robotic/automated surgical technologies).

The step 1110 is shown to include a sub-step 1113, determining if the position and function of the implant are satisfactory, according to an exemplary method shown and described in FIG. 15. The sub-step 1113 may be dependent on data, manipulation, and other outcomes of the sub-step 1112. For example, if the implant is measured to be in the correct place relative to anatomic features/landmarks and/or the one or more estimated/determined significant points, a physician may deem that the position and function of the implant are satisfactory. A physician may also account for a ROM when manipulating the joint and/or implant. However, if the implant is measured to be incorrectly placed (for example, does not meet the criteria mentioned above), a physician may elect to perform further testing or collect additional data, for example.

The step 1110 is shown to include a sub-step 1114 determination of “yes” the position and function of the implant are satisfactory, or a sub-step 1115 determination of “no” the position and/or the function of the implant is/are not satisfactory, according to an exemplary method shown and described in FIG. 15. In the event that the determination “yes” is made, and the position and function of the implant are determined to be satisfactory, the step 1110 is complete with the implant position and function confirmed. However, in the event that the determination “no” is made and the position and/or function of the implant is/are not determined to be satisfactory, the position and function of the implant may be adjusted and/or sub-step 1111 may be repeated. In some aspects, this may include repositioning of the implant and/or other hardware. The step 1110 may then be repeated until the sub-step 1113 is completed and the determination “yes” is reached. However, as mentioned previously the step 1110 may include one or more sub-steps that may be repeated and/or be performed out of order. That is to say that if the determination “yes” was made and a physician subsequently questions the position and/or function of the implant, one or more sub-steps of the step 1110 may be repeated. In some aspects, a determination of “no” may result to a return to the beginning of the process 700 and/or the sub-step 1111 for additional preparation.

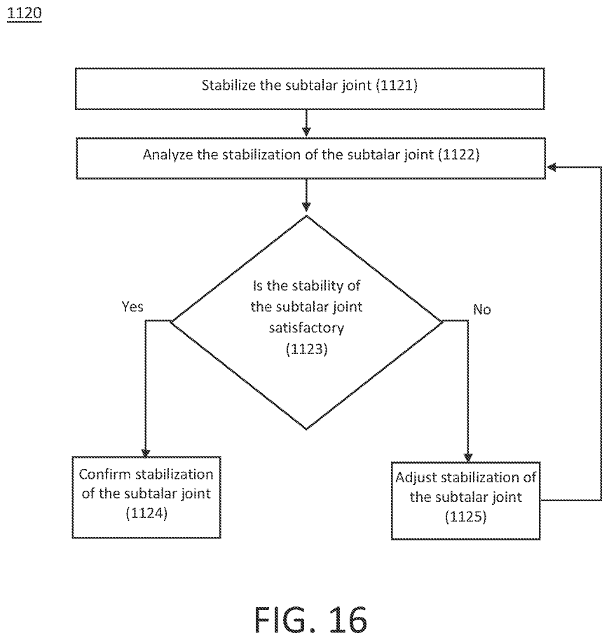

The process 1000 is shown to include a step 1120 stabilizing the subtalar joint, according to the exemplary method shown and described in FIGS. 12-16. The step 1120 may be performed one or more times and, in some aspects, may be an iterative process based on feedback received by the physician or other medical professional performing the step 320. Accordingly, the step 1120 is shown various sub-steps of stabilizing the subtalar joint, which are shown and described with reference to FIG. 16. As with step 1120, the sub-steps thereof may also be repeated one or more times, in part or in full. As with the process 1000, one or more sub-steps of step 1120 may be repeated, skipped, performed in an alternate order, or may be substituted for another step not shown in the exemplary embodiment of the step 1120 of FIG. 16.

The step 1120 is shown to include a step 1121 stabilizing the subtalar joint, according to the exemplary method shown and described in FIG. 16. The sub-step 1121 may include incorporating one or more imaging technologies including but not limited to those shown and described previously in order to collect data directed to stability of the subtalar joint. In some aspects, the stability of the subtalar joint may be largely dependent on various positioning, tensioning, and balancing of forces present in various soft tissue structures adjacent the subtalar joint (e.g., ligaments, tendons, muscles, etc.). In some aspects, the sub-step 1121 may include implementing one or more instruments in order to manipulate one or more soft tissue structures and/or measure forces present in the aforementioned soft tissue structures. For example, one or more instruments may be provided in conjunction with the implant and other components with said one or more instruments configured to be implemented in order to manipulate (e.g., reposition, shift, etc.) soft tissue. In some aspects, the sub-step 1121 may also include balancing of soft tissues to address deformities, trauma, or degeneration present at or near the subtalar joint.

The step 1120 is shown to include a sub-step 1122 analyzing the stabilization of the subtalar joint, according to the exemplary method shown and described in FIG. 16. The sub-step 1122 may include the implementation of imaging technology the same as and/or similar to that mentioned with respect to the sub-step 1121 in order to analyze, measure, and collect data indicative of one or more forces or positions of the soft tissue structures adjacent the subtalar joint. In some aspects, instruments provided in a kit or system with the implant may be implemented in order to facilitate the analysis of the stability of the subtalar joint. Further, analyzing the stability of the subtalar joint may include manipulation of the subtalar joint (including the implant) so as to measure and observe behavior of soft tissue structures with respect to movement of the implant. In some aspects, the physician may have a criteria (e.g., forces, positions, range of motion, etc.) which the various soft tissue structures must satisfy.

The step 1120 is shown to include a sub-step 1123 determining if the stability of the subtalar joint is satisfactory, according to the exemplary method shown and described in FIG. 16. The sub-step 1123 may be dependent on data, manipulation, and other outcomes of the sub-step 1122. For example, if the stability of the subtalar joint (e.g., soft tissue structures) is measured to meet a criteria relative to anatomic features/landmarks and/or the one or more estimated/determined significant points, a physician may deem that the stability of the subtalar joint is satisfactory. A physician may also account for range of motion when manipulating the joint and surrounding soft tissue structures. However, if the subtalar joint is measured to be unstable and/or incorrectly balanced (for example, does not meet the criteria mentioned above), a physician may elect to perform further testing or collect additional data and/or repeat necessary sub-steps to establish and confirm stability. .

The step 1120 is shown to include a sub-step 1124 determination of “yes” the stabilization of the subtalar joint is satisfactory, or a sub-step 1125 determination of “no” the stabilization of the subtalar joint is not satisfactory, according to an exemplary method shown and described in FIG. 16. In the event that the determination “yes” is made, and the stabilization of the subtalar joint is determined to be satisfactory, the step 1120 is complete with the stabilization of the subtalar joint confirmed. However, in the event that the determination “no” is made and the stabilization of the subtalar joint is not determined to be satisfactory, the stabilization of the subtalar joint be adjusted and/or sub-step 1121 may be repeated. In some aspects, this may include manipulation of one or more of the soft tissue structures relative to the subtalar joint/implant and/or the calcaneum 100 and/or the talus 200. The step 1120 may then be repeated until sub-step 1123 is completed and the determination “yes” is reached. However, as mentioned previously, step 1120 may include one or more sub-steps that may be repeated and/or be performed out of order. That is to say that if the determination “yes” was made and a physician subsequently questions the stability of the subtalar joint, one or more sub-steps of the step 1120 may be repeated (and/or various steps of the process 1000). In some aspects, a determination of “no” may result to a return to the beginning of the process 1000 and/or the step 1010 for additional preparation.

The process 1000 is shown to include a step 1130 restoring any necessary soft tissue structures and repairing the fibula, according to the exemplary method shown and described in FIGS. 12-16. The step 1130, in some aspects, be dependent on the steps 1010, 1020, and 1030. Depending on where the at least one incision was made and the soft tissue structures that were encountered/contacted in steps 1020 and 1030, similar and/or the same soft tissue structures may require restoration after the subtalar implant has been placed. For example, if the one or more incisions made included a lateral incision, a physician may restore one or more of the calcaneofibular ligament, the anterior talofibular ligament, and/or the extensor digitorium brevis. Similarly, if the one or more incisions made included a posterior incision, a physician may repair at least a portion of the Achilles and/or other tendons. In some aspects, a physician may implement various plates, fasteners, wedges, anchors, internal sutures, or other hardware/implants in performing any necessary repairs. Further, in some aspects, the physician may reference preoperative and/or intraoperative imaging in performing the fibular repair of step 1130.

The process 1000, as well as the various sub-steps included therein (and referred to according to steps 1010, 1090, 1100, and 1110) may also be performed/implemented in conjunction with a patient-specific implant (PSI) as well as other components of a PSI system (e.g., imaging technology, software, jigs, cut guides, instrumentation, etc.). For example, one or more sets of imaging data (e.g., collected using one or more of the imaging technologies mentioned herein) may be used to collect patient data prior to a subtalar arthroplasty procedure (e.g., the process 1000). This data may then be used to create (e.g., 3-D print, machine, etc.) one or more PSI components such as those mentioned previously. For example, one or more components of a subtalar joint implant may include a geometry that is based on the patient data collected prior to the subtalar arthroplasty procedure. Similarly, the steps 1070, 1090 of making one or more primary cuts in the calcaneum and talus, respectively, may include the use of one or more PSI cut guides and/or jigs. Such cut guides/jigs may be created based on the patient data collected and configured to guide one or more cuts so as to access and/or avoid various anatomical features. It should also be noted that PSI instrumentation may also be implemented in one or more of the steps of the process 1000.

The process 1000 is shown to include a step 1140 closing the at least one incision, according to the exemplary method shown and described in FIGS. 12-16. In some aspects, step 1140 may include closing one or more incisions using sutures or other fixation members and/or adhesives. Antibacterial agents may also be introduced at or near the incision in step 1140. The step 1140 is dependent upon the at least one incision made in the step 1010. In some aspects, only one incision was made in step 1010 and, thus, only one incision requires closing in step 1140. However, in other aspects multiple incisions may be made in step 1010 and accordingly multiple incisions require closing in step 1140.

As stated previously herein, the steps and sub-steps of the processes 700 and 1000 as shown and described herein may be omitted, modified, repeated, performed in an alternate order or otherwise adapted depending on circumstances of a patient, implant, procedure, or other circumstances. Additionally, the steps and sub-steps of the processes 700 and 1000 are understood to be cross-compatible. That is to say that, in some aspects, the process 1000 may incorporate the fibular osteotomy of the process 700, or the process 700 may incorporate the repositioning and temporary fixation of the talus of the process 1000. Additionally, any identified points, landmarks, planes, radii, geometric shapes/volumes, cut lines, or other significant/critical points described as identified herein may be applicable to the process 700 and/or the process 1000 (in addition to other processes not shown or described herein).

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. A method for performing an arthroplasty of a subtalar joint, the method comprising:
making at least one incision adjacent the subtalar joint;
accessing at least a portion of the subtalar joint, wherein the subtalar joint comprises a joint space defined superiorly by a first articular surface and inferiorly by a second articular surface;
preparing at least one of the first articular surface and the second articular surface to form a first interfacing surface and a second interfacing surface;
identifying at least one landmark on at least one of the first interfacing surface and the second interfacing surface;
determining at least one significant point on at least one of the first interfacing surface and the second interfacing surface relative to the at least one landmark;
placing at least one trial implant at least partially within the joint space;
securing an implant such that the implant is positioned substantially inferior relative to the first interfacing surface and substantially superior relative to the second interfacing surface;
stabilizing the subtalar joint; and
closing the at least one incision.

2. The method of claim 1, further comprising positioning the trial implant such that at least a portion of the trial implant contacts the first and second interfacing surfaces.

3. The method of claim 2, further comprising:
placing a first implant trial at least partially within the joint space; and
placing a second implant trial at least partially within the joint space;
wherein the implant trial comprises a first size and the second implant trial comprises a second size, wherein the second size is different from the first size.

4. The method of claim 1, further comprising performing a fibular osteotomy to access at least a portion of the at least one of the subtalar joint and the joint space.

5. The method of claim 1, further comprising analyzing a first position and a first function of the implant after the implant has been placed within at least a portion of the joint space.

6. The method of claim 5, further comprising adjusting the implant within at least a portion of the joint space such that the implant comprises a second position and a second function, wherein the second position and function are different than the first position and function.

7. The method of claim 1, further comprising temporarily fixing at least one of a calcaneum and a talus before preparing at least one of the first articular surface and the second articular surface.

8. The method of claim 1, wherein stabilizing the subtalar joint comprises manipulating one or more soft tissue structures adjacent the subtalar implant after the implant has been placed and fixated at least partially within the joint space to reach a first stability.

9. The method of claim 9, further comprising confirming stability of the subtalar joint.

11. The method of claim 1, wherein securing the implant comprises placing at least a first implant component adjacent the first interfacing surface and placing a second implant component adjacent the second interfacing surface.

12. The method of claim 11, further comprising placing a third implant component at least partially within the joint space such that the third implant component is positioned substantially inferior relative to the first implant component and substantially superior relative to the second implant component.

13. The method of claim 1, wherein the at least one significant point is at least one center of rotation disposed on the second interfacing surface.

14. The method of claim 1, further comprising aligning one or more components of the subtalar joint.

15. The method of claim 14, wherein aligning the subtalar joint comprises manipulating at least one of a talus and a calcaneum such that at least a head of the talus is disposed substantially superior relative to at least a portion of the calcaneum.

16. The method of claim 1, wherein the at least one landmark comprises one or more facets substantially disposed on a superior surface of the calcaneum.

17. The method of claim 1, wherein the first interfacing surface is an inferior surface of the talus, and the second interfacing surface is a superior surface of the calcaneum.

18. The method of claim 1, further comprising manipulating one or more soft tissue structures.

19. A method for performing arthroplasty of a subtalar joint, the method comprising:
performing a fibular osteotomy to access at least a portion of the subtalar joint;
identifying at least one landmark on at least one of an inferior surface and a superior surface;
determining at least one center of rotation disposed on at least one of the inferior and superior surfaces;
placing at least one trial implant adjacent the inferior and superior surfaces; and
securing an implant such that the implant is positioned substantially between the inferior and superior surfaces.

20. A method for performing an arthroplasty of a subtalar joint, the subtalar joint comprising a first surface and a second surface, the method comprising:
identifying at least one landmark on at least one of the first surface and the second articular surface;
determining at least one significant point in relation to the at least one landmark; and
securing an implant between the first and second surfaces in a first position, wherein the first position is based on a position of the at least one significant point.
